(19) United States
(12) Patent Application Publication
(10) Pub. No.: US 2026/0232932 A1
(43) Pub. Date: Aug. 13, 2026

(54) DUAL STRIP DRY POWDER INHALER AND MANIFOLDS FOR USE THEREIN

(71) Applicant: TRANSPIRE BIO INC., Weston, FL (US)

(72) Inventors: Thomas Alexander EARWAKER, Leamington Spa (GB); Zoe Georgina ARMSTRONG, Leamington Spa (GB); Michael Ray NICHOLSON-LEWIS, Leamington Spa (GB); Matthew Meredith JONES, Warwick (GB); Jianye WANG, St. Albans (GB)

(21) Appl. No.: 19/153,787

(22) PCT Filed: Feb. 1, 2024
(86) PCT No.: PCT/US2024/014013

§ 371 Date: Aug. 5, 2025

Related U.S. Application Data
(60) Provisional application No. 63/483,385, filed on Feb. 6, 2023.

(51) Int. Cl. A61M 15/00

(52) U.S. Cl. CPC A61M 15/0051; A61M 15/0003; A61M 15/002; A61M 15/0026; A61M 15/0071; A61M 15/0091; A61M 2202/0007; A61M 2202/064; A61M 2205/583; A61M 2206/10

(57) ABSTRACT

Inhaler devices for simultaneously delivering dry powdered medicament from two blister strips. Manifolds for use in the inhaler devices are configured to guide airflow towards two opened blister pockets of the two blister strips for accessing the dry powdered medicament contained therein, and subsequently guide the powdered medicament to a mouthpiece of the inhaler device for inhalation by a patient.

### CROSS REFERENCE OF RELATED APPLICATIONS

This application claims the benefit of U.S. Provisional Patent Application No. 63/483385, filed Feb. 6, 2023, which is hereby incorporated by reference in its entirety for all purposes.

### FIELD OF THE INVENTION

The invention relates in general to an inhaler device, and more particularly to manifolds for use in an inhaler device configured to simultaneously dispense dry power medicament from two blister strips.

### BACKGROUND OF THE INVENTION

Medicaments may be administered to a patient by inhalation using dry powder inhaler devices. Such devices are often used for the treatment and prophylaxis of respiratory diseases, including but not limited to asthma and chronic obstructive pulmonary disease (COPD). A dry powder inhaler device may include a blister strip containing several discrete doses of powdered medicament. Such devices typically contain a mechanism such as piercing means for accessing a medicament dose by opening one or more blister pockets. The powdered medicament can then be accessed by the device and inhaled via the device by the user.

Inhaler devices of the type described above suitably include a component, generally referred to as a manifold, for guiding airflow towards one or more opened blister pocket(s) and the powdered medicament contained therein, and subsequently guiding the powdered medicament to a mouthpiece for inhalation by a patient. The characteristics of the manifold are important to ensure liberation of the dry powder medicament and to subsequently guide the dry liberated powder medicament to the mouthpiece. Embodiments hereof relate to improved manifolds for use in a dry powder inhaler device.

### BRIEF SUMMARY OF THE INVENTION

According to a first embodiment hereof, the present disclosure provides a manifold for use in an inhaler device for the simultaneous delivery of powdered medicament from an open blister pocket of each of a first blister strip and a second blister strip. The manifold includes a body defining a first atrium that includes an atrium inlet, a first atrium outlet and a second atrium outlet, a second atrium that includes an atrium inlet, a first atrium outlet and a second atrium outlet, and at least one stack that includes at least one stack outlet, a first stack inlet, a second stack inlet, a third stack inlet, and a fourth stack inlet. The first atrium outlet of the first atrium is in fluid communication with the first stack inlet to define a first diversion airflow path of the manifold. The first atrium outlet of the second atrium is in fluid communication with the third stack inlet to define a second diversion airflow path of the manifold. The second atrium outlet of the first atrium is in fluid communication with the second stack inlet to define a first entrainment airflow path associated with an open blister pocket of the first blister strip. The second atrium outlet of the second atrium is in fluid communication with the fourth stack inlet to define a second entrainment airflow path associated with an open blister pocket of the second blister strip.

In an aspect of the first embodiment, and in combination with any other aspects herein, the disclosure provides that the at least one stack is a single stack and the at least one stack outlet is a single stack outlet. The first diversion airflow path, the second diversion airflow path, the first entrainment airflow path, and the second entrainment airflow path mix together within the single stack.

In an aspect of the first embodiment, and in combination with any other aspects herein, the disclosure provides that each of the first and second diversion airflow paths is configured to disrupt each of the first and second entrainment airflow paths and break up powdered medicament carried thereby.

In an aspect of the first embodiment, and in combination with any other aspects herein, the disclosure provides that the at least one stack includes a first stack and a second stack. The first diversion airflow path and the first entrainment airflow path mix together in the first stack, and the second diversion airflow path and the second entrainment airflow path mix together in the second stack.

In an aspect of the first embodiment, and in combination with any other aspects herein, the disclosure provides that the first stack and the second stack are separated by a divider wall such that mixing of the first entrainment airflow path and the second entrainment airflow path is prevented within the manifold.

In an aspect of the first embodiment, and in combination with any other aspects herein, the disclosure provides that the first stack and the second stack are separated by a divider wall such that mixing of the first entrainment airflow path and the second entrainment airflow path is prevented along a height of the divider wall.

In an aspect of the first embodiment, and in combination with any other aspects herein, the disclosure provides that the height of the divider wall is less than a height of the first and second stacks such that mixing of the first entrainment airflow path and the second entrainment airflow path is permitted within the manifold downstream of the divider wall.

In an aspect of the first embodiment, and in combination with any other aspects herein, the disclosure provides that the first diversion airflow path is configured to disrupt the first entrainment airflow path and break up powdered medicament carried thereby. The second diversion airflow path is configured to disrupt the second entrainment airflow path and break up powdered medicament carried thereby.

In an aspect of the first embodiment, and in combination with any other aspects herein, the disclosure provides that the first atrium and the second atrium are separated by a divider wall such that the first atrium is not in fluid communication with the second atrium.

In an aspect of the first embodiment, and in combination with any other aspects herein, the disclosure provides that the first atrium and the second atrium are substantially similar in volume.

In an aspect of the first embodiment, and in combination with any other aspects herein, the disclosure provides that first atrium and the second atrium are different in volume.

In an aspect of the first embodiment, and in combination with any other aspects herein, the disclosure provides that first atrium and the second atrium are disposed on opposing sides of the body of the manifold.

In an aspect of the first embodiment, and in combination with any other aspects herein, the disclosure provides that first atrium and the second atrium are disposed adjacent to each other on a single side of the body of the manifold.

In an aspect of the first embodiment, and in combination with any other aspects herein, the disclosure provides that each of the second atrium outlet of the first atrium and the second atrium outlet of the second atrium includes a grill spanning it.

In an aspect of the first embodiment, and in combination with any other aspects herein, the disclosure provides that the first diversion airflow path, the second diversion airflow path, the first entrainment airflow path, and the second entrainment airflow path are respective airflow portions of an inhalation airflow which is drawn into the manifold through the atrium inlet of each of the first and second atriums when an inhalation force is applied at the at least one stack outlet.

According to a second embodiment hereof, the present disclosure provides a manifold for use in an inhaler device for the simultaneous delivery of powdered medicament from an open blister pocket of each of a first blister strip and a second blister strip. The manifold includes a body defining a first atrium that includes an atrium inlet and at least one atrium outlet, a second atrium that includes an atrium inlet, a first atrium outlet and a second atrium outlet, and a stack that includes a stack outlet, a first stack inlet, a second stack inlet, and a third stack inlet. The at least one atrium outlet of the first atrium is in fluid communication with the first stack inlet to define a first diversion airflow path of the manifold. The first atrium outlet of the second atrium is in fluid communication with the second stack inlet to define a first entrainment airflow path associated with an open blister pocket of the first blister strip. The second atrium outlet of the second atrium is in fluid communication with the third stack inlet to define a second entrainment airflow path associated with an open blister pocket of the second blister strip.

In an aspect of the second embodiment, and in combination with any other aspects herein, the disclosure provides that the first diversion airflow path, the first entrainment airflow path, and the second entrainment airflow path mix together within the stack.

In an aspect of the second embodiment, and in combination with any other aspects herein, the disclosure provides that the first diversion airflow path is configured to disrupt each of the first and second entrainment airflow paths and break up powdered medicament carried thereby.

In an aspect of the second embodiment, and in combination with any other aspects herein, the disclosure provides that the first atrium and the second atrium are separated by a divider wall such that the first atrium is not in fluid communication with the first atrium.

In an aspect of the second embodiment, and in combination with any other aspects herein, the disclosure provides that the first atrium and the second atrium are substantially similar in volume.

In an aspect of the second embodiment, and in combination with any other aspects herein, the disclosure provides that the first atrium and the second atrium are different in volume.

In an aspect of the second embodiment, and in combination with any other aspects herein, the disclosure provides that the first atrium and the second atrium are disposed on opposing sides of the body of the manifold.

In an aspect of the second embodiment, and in combination with any other aspects herein, the disclosure provides that the first atrium and the second atrium are disposed adjacent to each other on a single side of the body of the manifold.

In an aspect of the second embodiment, and in combination with any other aspects herein, the disclosure provides that each of the first atrium outlet of the second atrium and the second atrium outlet of the second atrium includes a grill spanning it.

In an aspect of the second embodiment, and in combination with any other aspects herein, the disclosure provides that the first diversion airflow path, the first entrainment airflow path, and the second entrainment airflow path are respective airflow portions of an inhalation airflow which is drawn into the manifold through the atrium inlet of each of the first and second atriums when an inhalation force is applied at the stack outlet.

In an aspect of the second embodiment, and in combination with any other aspects herein, the disclosure provides that the at least one atrium outlet of the first atrium is a single outlet.

In an aspect of the second embodiment, and in combination with any other aspects herein, the disclosure provides that the at least one atrium outlet of the first atrium includes a first atrium outlet and a second atrium outlet. The first atrium outlet of the first atrium is in fluid communication with the first stack inlet to define the first diversion airflow path of the manifold, and the second atrium outlet is in fluid communication with a fourth stack inlet to define a second diversion airflow path of the manifold.

### BRIEF DESCRIPTION OF DRAWINGS

The foregoing and other features and advantages of the invention will be apparent from the following description of embodiments hereof as illustrated in the accompanying drawings. The accompanying drawings, which are incorporated herein and form a part of the specification, further serve to explain the principles of the invention and to enable a person skilled in the pertinent art to make and use the invention. The drawings are not to scale.

FIG. 1A is a front view of an inhaler device according to an embodiment hereof, wherein a mouthpiece cover of the inhaler device is in a closed position.

FIG. 1B is a rear view of the inhaler device of FIG. 1A, wherein the mouthpiece cover of the inhaler device is in the closed position.

FIG. 1C is a front view of the inhaler device of FIG. 1A, wherein the mouthpiece cover of the inhaler device is in an open position.

FIG. 1D is a graph that illustrates an estimated actuation force profile for the inhaler device and mouthpiece cover as depicted in FIGS. 1A-1C (in the dotted line), compared to an estimated actuation force profile for an inhaler device in which a dispensing mechanism is not actuated for an initial period of travel of a mouthpiece cover (in the solid line).

FIG. 2 is a perspective view of two blister strips for use within the inhaler device of FIG. 1A.

FIG. 3A is a front view of the inhaler device of FIG. 1A, wherein the mouthpiece cover of the inhaler device is in the open position and a housing of the inhaler device is removed for sake of illustration only.

FIG. 3B is a sectional perspective view of a portion of the inhaler device of FIG. 1A illustrating a portion of the airflow path through the inhaler device of FIG. 1A.

FIG. 4A is a front view of the inhaler device of FIG. 1A, wherein the mouthpiece cover of the inhaler device is in the open position and a portion of the housing of the inhaler device is removed for sake of illustration only.

FIG. 4B is a rear view of the inhaler device of FIG. 1A, wherein the mouthpiece cover and the housing of the inhaler device are removed for sake of illustration only.

FIG. 5 is a front view of a ratchet mechanism of the inhaler device of FIG. 1A, wherein the ratchet gear is removed from the inhaler device for sake of illustration only.

FIG. 5A illustrates the ratchet mechanism of FIG. 5 when the mouthpiece cover is in the closed position.

FIG. 5B illustrates the ratchet mechanism of FIG. 5 when the mouthpiece cover is in the open position.

FIG. 6 is a perspective view of the mouthpiece cover and a portion of a dispensing subassembly of the inhaler device of FIG. 1A, wherein the mouthpiece cover and dispensing subassembly are removed from the inhaler device for sake of illustration only.

FIG. 7A is a perspective view of a counter subassembly of the inhaler device of FIG. 1A, wherein the counter assembly is removed from the inhaler device for sake of illustration only.

FIG. 7B is a front view of the counter subassembly of FIG. 7A.

FIG. 7C is a sectional view of the counter subassembly of FIG. 7A, taken along line C-C of FIG. 7B.

FIG. 7D is a sectional view of the counter subassembly of FIG. 7A, taken along line D-D of FIG. 7C.

FIG. 8A is a schematic illustration of a tensioning mechanism of the inhaler device of FIG. 1A, wherein the tensioning mechanism is shown at a beginning of the device life.

FIG. 8B is a schematic illustration of a tensioning mechanism of the inhaler device of FIG. 1A, wherein the tensioning mechanism is shown near an end of the device life.

FIG. 9A is a perspective view of a tensioning mechanism of the inhaler device of FIG. 1A, wherein the tensioning mechanism is removed from the inhaler device for sake of illustration only.

FIG. 9B is a sectional view of the tensioning mechanism of FIG. 9A.

FIG. 9C is a cross-sectional view of the tensioning mechanism of FIG. 9A.

FIG. 9D is a perspective exploded view of a base and a nut of the tensioning mechanism of FIG. 9A.

FIG. 9E is a series of sectional views of the tensioning mechanism of FIG. 9A illustrating the movement of the nut during operation of the inhaler device.

FIG. 10 is a perspective view of a manifold of the inhaler device of FIG. 1A, wherein the manifold is removed from the inhaler device for sake of illustration only.

FIG. 10A is a sectional view taken along line A-A of FIG. 10.

FIG. 11 is a perspective view of the manifold of FIG. 10 positioned adjacent to inlet vents of the housing of the inhaler device of FIG. 1A.

FIG. 12 is another perspective view of a manifold of the inhaler device of FIG. 1A, wherein the manifold is removed from the inhaler device for sake of illustration only.

FIG. 13A is a schematic view illustrating an airflow path through the manifold of FIG. 12.

FIG. 13B is a schematic flowchart illustrating the airflow path through the manifold of FIG. 12.

FIG. 14 is a perspective view of a manifold according to another embodiment hereof, wherein the manifold is removed from an inhaler device for sake of illustration only.

FIG. 15 is a perspective view of a manifold according to another embodiment hereof, wherein the manifold is removed from an inhaler device for sake of illustration only.

FIG. 16 is a schematic view illustrating an airflow path through the manifold of FIG. 15.

FIG. 17 is a schematic flowchart illustrating the airflow path through the manifold of FIG. 15.

FIG. 18A is a perspective front view of a manifold according to another embodiment hereof, wherein the manifold is removed from an inhaler device for sake of illustration only.

FIG. 18B is a perspective rear view of the manifold of FIG. 18A.

FIG. 19 is a schematic view illustrating an airflow path through the manifold of FIG. 18A.

FIG. 20 is a schematic flowchart illustrating the airflow path through the manifold of FIG. 18A.

FIG. 21A is a perspective view of the manifold of FIG. 18A positioned adjacent to inlet vents of a housing of an inhaler device.

FIG. 21B is a sectional perspective view of a portion of the manifold of FIG. 18A illustrating a portion of the airflow path therethrough.

FIG. 22A is a perspective front view of a manifold according to another embodiment hereof, wherein the manifold is removed from an inhaler device for sake of illustration only.

FIG. 22B is a perspective rear view of the manifold of FIG. 22A.

FIG. 23 is a schematic view illustrating an airflow path through the manifold of FIG. 22A.

FIG. 24 is a schematic flowchart illustrating the airflow path through the manifold of FIG. 22A.

FIG. 25 is a perspective view of the manifold of FIG. 22A illustrating the airflow path therethrough.

FIG. 26 is a perspective view of a manifold according to another embodiment hereof, wherein the manifold is removed from an inhaler device for sake of illustration only.

### DETAILED DESCRIPTION OF EMBODIMENTS OF THE INVENTION

Specific embodiments of the present invention are now described with reference to the figures, wherein like reference numbers indicate identical or functionally similar elements. The following detailed description is merely exemplary in nature and is not intended to limit the invention or the application and uses of the invention. Although the description of the invention is in the context of dry powder inhaler devices, the invention may also be used in other applications where it is deemed useful. Furthermore, there is no intention to be bound by any expressed or implied theory presented in the preceding technical field, background, brief summary or the following detailed description.

Embodiments hereof relate to inhaler devices configured to deliver powdered medicament from two blister strips, and more particularly, relate to manifolds for use therein for guiding airflow towards two opened blister pockets of the blister strips and accessing the powdered medicament contained therein, and subsequently guiding the powdered medicament to a mouthpiece of the inhaler device for inhalation by a patient. The manifolds described herein are intended for use in a dry powder inhaler device in which the powdered medicament dose is intended to be entrained in an airflow generated by inhalation. Manifolds herein are configured to direct the airflow or percentages of the airflow to help the successful transfer of the powdered medicament dose from the two open blister pockets to the mouthpiece of the inhaler device. Successful transfer of the powdered medicament dose is achieved when the flow resistance of the air pathway is sufficiently low, the entrainment of the powdered medicament dose from the two open blister pockets is sufficiently high, and there is sufficient deagglomeration of the powdered medicament prior to reaching the outlet(s) of the manifold. Transfer of the powdered medicament dose with a sufficiently small particle size is critical, since the delivery of the powdered medicament to the user's lungs via the mouthpiece is the primary function of the inhaler device.

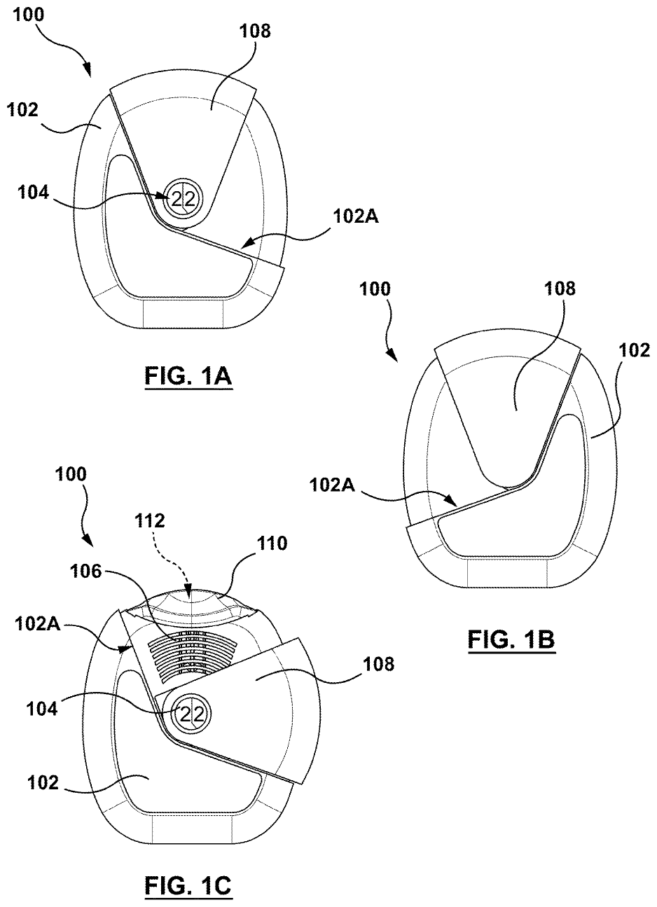

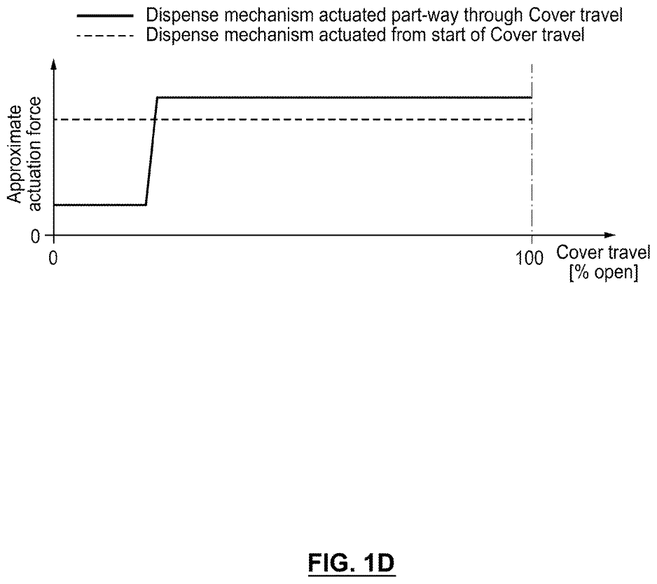

FIGS. 1A, 1B, and 1C illustrate an inhaler device 100 according to an embodiment hereof. The inhaler device 100 includes a housing 102 and a mouthpiece cover 108. The housing 102 includes a display window 104 through which a number is displayed, the number indicating the number of remaining doses of the inhaler device 100. The housing 102 also includes a plurality of openings or inlet vents 106 formed through a sidewall of the housing 102. As will be described in more detail herein, air from outside of the inhaler device 100 is drawn into the interior of the inhaler device 100 via the inlet vents 106 upon inhalation, by a user, at a mouthpiece 110. In FIGS. 1A and 1B, the mouthpiece cover 108 of the inhaler device 100 is in a closed position in which the mouthpiece cover 108 covers or extends over the mouthpiece 110. The mouthpiece cover 108 is in an open position in FIG. 1C such that the mouthpiece 110 is exposed and available to the user. The user can inhale the powdered medicament through the mouthpiece 110 only when the mouthpiece cover 108 is in the open configuration. The mouthpiece 110 includes a central outlet or opening 112 which permits delivery of the powdered medicament contained within the inhaler device 100 to the user via inhalation.

In the closed position of FIGS. 1A and 1B, the mouthpiece 110 and the inlet vents 106 are covered by the mouthpiece cover 108. When a user wishes to inhale a medicament dose from the inhaler device 100, the mouthpiece cover 108 is moved from the closed position of FIGS. 1A and 1B to the open position of FIG. 1C. In the open position, the mouthpiece cover 108 is rotated or moved relative to the housing 102 such that the mouthpiece 110 and the inlet vents 106 are fully exposed and no longer covered by any portion of the mouthpiece cover 108. Since users are instructed to cover the central opening 112 of the mouthpiece 110 with their mouth during inhalation of the powdered medicament, the mouthpiece cover 108 protects the mouthpiece 110 when the inhaler device 100 is not in use to prevent contamination of the airflow passageway of the inhaler device 100 by unwanted particles, which could otherwise have a negative impact on user experience and/or dose delivery. As will be described in more detail herein, the movement of the mouthpiece cover 108 from the closed position to the open position actuates a dispensing mechanism in the inhaler device 100 to make a medicament dose available for inhalation and further actuates a counter mechanism in the inhaler device 100 to decrease the number of remaining doses shown in the display window 104 by one unit. Thus, the mouthpiece cover 108 functions to protect the central opening 112 of the mouthpiece 110 and also operates the dispensing and counter mechanisms of the inhaler device 100. Only a single operating step, i.e., movement of the mouthpiece cover 108, is required by the user to actuate the inhaler device 100 for each dose.

In an embodiment, a mouthpiece cover 108 may be rotated between 85 and 105 degrees by the user to expose a mouthpiece 110 and inlet vents 106. In designing the inhaler device 100, it is important to ensure that a force required to actuate the mouthpiece cover 108 is sufficiently low to allow easy operation by users with a range of abilities. In general, when a distance of travel of the mouthpiece cover 108 is longer, a more favorable mechanical advantage is realized in a dispensing mechanism of the inhaler device, which results in a lower actuation force being needed to rotate the mouthpiece cover 108 and actuate the inhaler device 100 for each dose. However, from an ergonomics perspective, a shorter distance of travel of the mouthpiece cover 108 may avoid the user having to change grip during actuation. A shorter travel distance of the mouthpiece cover 108 also results in a larger area of the housing 102 for the user to grip during actuation, since there is a smaller area of the housing 102 traversed by the mouthpiece cover 108. A shorter travel of the mouthpiece cover 108 also allows more space for other features of the inhaler device 100, and/or allows a size of the inhaler device 100 to be minimized. In an embodiment, a cover travel between 90 and 100 degrees may provide an optimal balance between the above noted factors. As shown in FIGS. 1A-1C, the housing 102 includes an integral flange or step 102A formed thereon that controls or limits rotational movement of the mouthpiece cover 108 to the desired range.

The force profile over the travel of the mouthpiece cover 108 will affect the user experience and tactile feedback given by the inhaler device 100. Maintaining a relatively consistent or constant actuation force over the travel of the mouthpiece cover 108 is also desirable to avoid incorrect usage or confusion. Using the full travel of the mouthpiece cover 108 for operating the dispensing mechanism is expected to make the actuation force profile more consistent and help to mitigate the risk of misuse of the inhaler device 100. For example, in inhaler devices unlike the present invention in which a dispensing mechanism is not actuated for an initial period of the mouthpiece cover travel, an actuation force is relatively low in this initial period. At the point in which the dispensing mechanism is actuated, the actuation force of the mouthpiece cover increases. Thus in inhalers unlike the present invention, the actuation force of the mouthpiece cover significantly increases midway through the total travel of the mouthpiece cover, and a user may incorrectly perceive this change as tactile feedback suggesting that the mouthpiece cover is sufficiently open to take a dose. Stated another way, a non-constant actuation force profile may be confusing for a user in terms of the tactile feedback and could cause incorrect usage of the device. FIG. 1D illustrates an estimated actuation force profile, represented by a dashed line, for the inhaler device 100 according to embodiments herein that has the mouthpiece cover 108, as compared to an estimated actuation force profile, represented by a solid line, for an inhaler device in which the dispensing mechanism is not actuated for an initial period of the mouthpiece cover travel as explained in the prior example. When a dispensing mechanism does not actuate at the beginning of the mouthpiece travel, there is a step in the actuation force profile as represented by the solid line in FIG. 1D. Conversely, when a dispensing mechanism actuates at the beginning of the mouthpiece travel as in the inhaler device 100, then the actuation force is generally constant or consistent and the peak actuation force is lower, as represented by the dashed line in FIG. 1D.

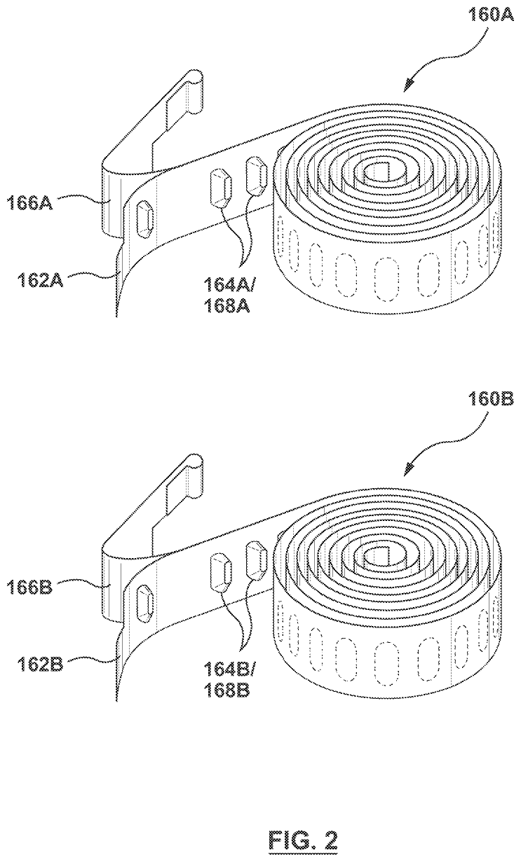

Inhaler device 100 is configured to simultaneously dispense dry powder medicaments from two blister strips. More particularly, with reference to FIG. 2, a first blister strip 160A and a second blister strip 160B are shown. Inhaler device 100 described herein is configured to simultaneously dispense medicament from each of the first and second blister strips 160A, 160B. Each blister strip 160A, 160B includes a bottom sheet 162A, 162B, respectively, which defines a series or plurality of individual blisters or pockets 164A, 164B thereon. Each pocket 164A, 164B is configured to contain a dose or portion thereof of dry powder or powdered medicaments 168A, 168B to be inhaled by a user. In an embodiment, the powdered medicament 168A is a different medicament than the powdered medicament 168B so that the inhaler device 100 is configured to simultaneously deliver two different powdered medicaments to the user. A top sheet 166A, 166B is hermetically bonded or sealed to the bottom sheet 162A, 162B, respectively, to close the pockets 164A, 164B, respectively and function as a lid for the pockets 164A, 164B to retain the powdered medicaments 168A, 168B therein. The hermetic sealing of the top sheets 166A, 166B is such that the bottom sheets 162A, 162B and the top sheets 166A, 166B are able to be peeled apart to open or uncover the pockets 164A, 164B for access to the powdered medicaments 168A, 168B therein. Each of the first and second blister strips 160A, 160B is sufficiently flexible to be wound into a roll.

As will be described in more detail herein, when a dispensing mechanism of the inhaler device 100 is actuated via movement of the mouthpiece cover 108, the top sheets 166A, 166B of the blister strips 160A, 160B, respectively, are peeled away from the bottom sheets 162A, 162B of the blister strips 160A, 160B, respectively, to open or uncover a pocket 164A, 164B of each blister strip and thereby expose the powdered medicaments 168A, 168B disposed therein. Upon inhalation via the mouthpiece 110, the user simultaneously inhales the powdered medicaments 168A, 168B from the opened pockets 164A, 164B of the blister strips 160A, 160B, respectively. The user thus receives a fixed metered dose of medicament powder of which the different medicament powders from the opened pockets 164A, 164B of the blister strips 160A, 160B, respectively, make up respective dose portions. Each blister strip 160A, 160B may be of the same size and/or contain the same dose amount (e.g., volume or mass) of powdered medicament or may be of different sizes and/or contain different dose amounts of powdered medicament.

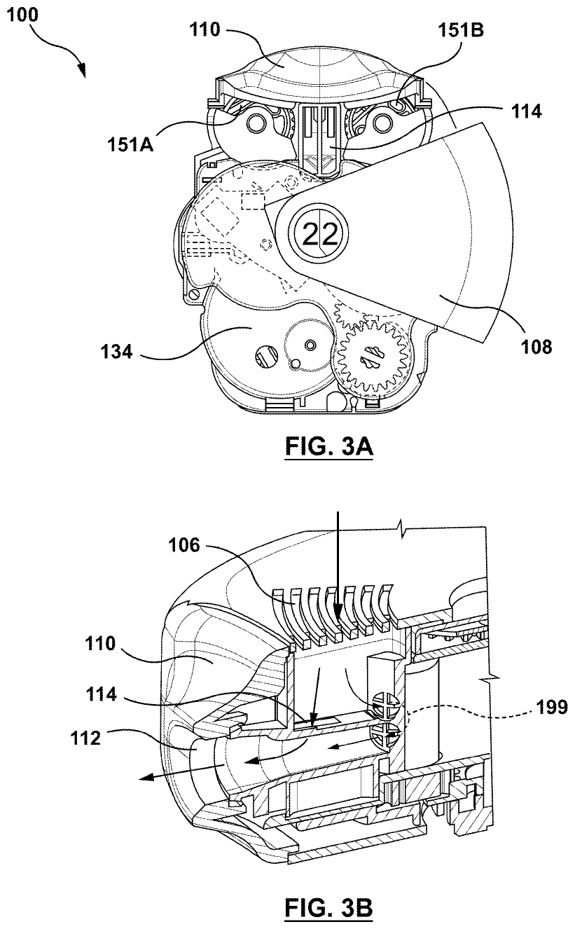

FIG. 3A is a front view of the inhaler device 100 with the mouthpiece cover 108 in the open position and the housing 102 removed for sake of illustration only. The inhaler device 100 includes a manifold 114 for directing airflow therethrough to entrain and deliver the powdered medicaments 168A, 168B from the blister strips 160A, 160B, respectively, to the user via the mouthpiece 110. The manifold 114 is in fluid communication with the mouthpiece 110 such that the powdered medicaments 168A, 168B may be delivered to the user through the central opening 112 of the mouthpiece 110. In addition to the manifold 114, the inhaler device 100 also includes a dispensing subassembly or mechanism 120, a counter subassembly or mechanism 134, and tensioning subassemblies or mechanisms 151A, 151B. When assembled, each of the manifold 114, the dispensing subassembly 120, the counter subassembly 134, and the tensioning mechanisms 151A, 151B reside or are disposed within the housing 102.

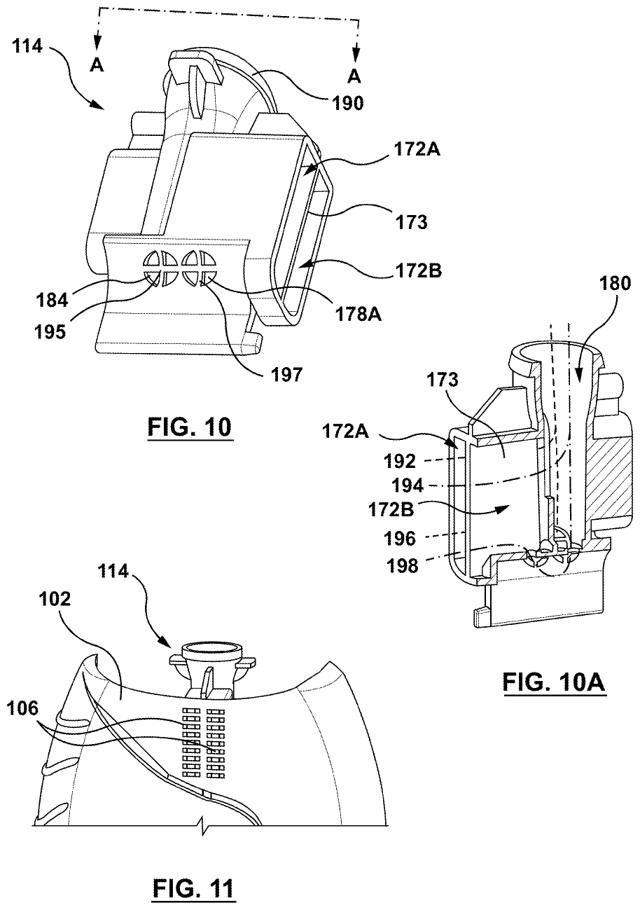

While the structure of the manifold 114 is described in detail herein with respect to FIGS. 10-14, the operation of the manifold 114 is introduced herein with reference to FIG. 3B. The manifold 114 defines the air pathway through the inhaler device 100. The manifold 114 fluidly connects the mouthpiece 110 to the first and second blister strips 160A, 160B. FIG. 3B is a sectional view taken through the manifold 114 to illustrate an airflow path therethrough for entrainment of medicament 168B of the second blister strip 160B. As explained with respect to FIGS. 10-14, the manifold 114 also defines an airflow path therethrough for entrainment of medicament 168A of the first blister strip 160A.

In use, the mouthpiece cover 108 is rotated by the user to expose the mouthpiece 110 and the inlet vents 106. Internally, within the inhaler device 100, rotating the mouthpiece cover 108 exposes the powdered medicaments 168A, 168B within a pocket 164A, 164B, respectively, of each of first and second blister strips 160A, 160B. To access the powdered medicaments 168A, 168B within the opened pockets 164A, 164B, the user breathes in or inhales through the mouthpiece 110. By covering the central opening 112 of the mouthpiece 110 with their mouth and inhaling, the user creates a pressure differential between the inlet vents 106 and the central opening 112 and causes air to travel through the manifold 114. The pressure differential causes external air (i.e., air from outside of the inhaler device 100) to enter the inhaler device 100 through the inlet vents 106, pass through the opened pockets 164A, 164B, and exit the inhaler device 100 through the central opening 112. The airflow pathways defined by the manifold 114 are designed such that when the user inhales, the exposed powdered medicaments 168A, 168B within the opened pockets 164A, 164B are picked up by the airstreams and delivered to the user as an orally inhaled combined medicament dose. As such, the user can simultaneously inhale one dose portion from each blister strip 160A, 160B.

As will be described in more detail herein with respect to FIGS. 10-14, the manifold 114 is configured to direct the inhalation airflow in a variety of ways to achieve airflow properties that are advantageous for effective delivery of the powdered medicaments. More particularly, as shown in FIG. 3B, the geometry of manifold 114 causes a portion of the inhalation airflow to enter and exit an opened pocket 164B of the second blister strip 160B (labeled with a dotted line 199 in FIG. 3B), while another portion of the inhalation airflow travels through a diversion or hole in the manifold 114 (which is described in more detail in FIGS. 10-14). The portion of inhalation airflow through the opened pocket 164B results in entrainment of the powdered medicament dose 168B in the airflow, and the portion of inhalation airflow through the diversion in the manifold 114 intersects the entrained airflow portion to break up the powdered medicament therein before exiting the manifold 114.

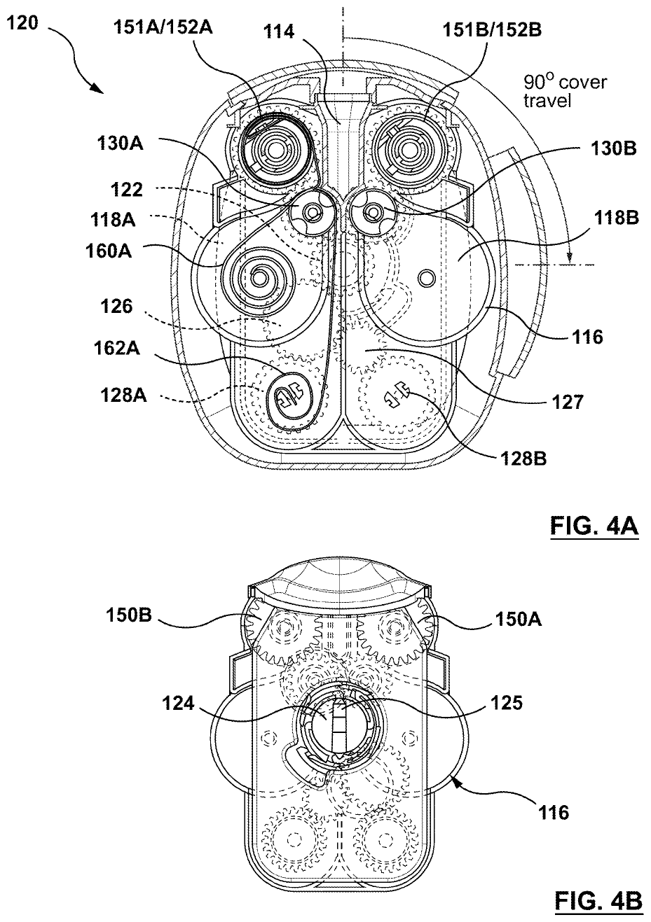

Turning now to FIGS. 4A and 4B, the dispensing subassembly 120 of the inhaler device 100 is described in more detail. The dispensing subassembly 120 is configured to advance each blister strip 160A, 160B and open a pocket 164A, 164B thereof each time the mouthpiece cover 108 is fully opened by the user. The first and second blister strips 160A, 160B are disposed within first and second compartments 118A, 118B within the housing 102. More particularly, compartments 118A, 118B are formed via an internal chassis 116 disposed within the housing 102. Via the dispensing subassembly 120, successive pockets 164A, 164B of each blister strip 160A, 160B are guided towards the manifold 114 which is disposed along a centerline, or disposed approximately along the centerline, of the inhaler device 100. When positioned at the manifold 114, a pocket 164A, 164B of each blister strip 160A, 160B has been opened and the powdered medicaments 168A, 168B within the opened pocket of each blister strip 160A, 160B is available for inhalation. The empty bottom sheets 162A, 162B and the top sheets 166A, 166B of the blister strips 160A, 160B are coiled up by the dispensing subassembly 120 as described herein. FIG. 4A is a front view of the inhaler device 100 with the mouthpiece cover 108 in the open position and a front half or portion of the housing 102 of the inhaler device 100 is removed for sake of illustration only. FIG. 4B is a rear view of the inhaler device 100 with the mouthpiece cover 108 and the housing 102 removed for sake of illustration only.

The dispensing subassembly 120 includes a central driver gear 122, a ratchet mechanism 124, a first idler or intermediate gear 126, a second idler or intermediate gear 127, first and second bottom sheet take-up gears 128A, 128B, first and second index gears 130A, 130B, and first and second top sheet take-up gears 150A, 150B. The first bottom sheet take-up gear 128A, the first index gear 130A, and the first top sheet take-up gear 150A are associated with advancement of the first blister strip 160A, while the second bottom sheet take-up gear 128B, the second index gear 130B, and the second top sheet take-up gear 150B are associated with advancement of the second blister strip 160B.

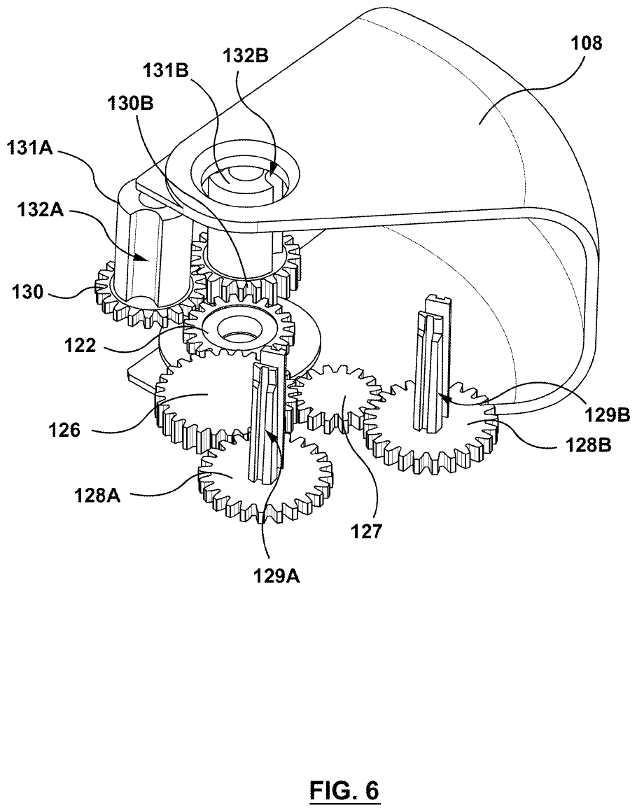

The first and second index gears 130A, 130B are attached to or integrally formed with first and second index spools 131A, 131B, respectively, as shown in FIG. 6. Each of first and second index spools 131A, 131B include a pair of recesses 132A, 132B, respectively, thereon. Each recess of the pair of recesses 132A, 132B is configured to receive a pocket 164A, 164B, respectively, of the blister strips 160A, 160B. Via rotation of the first and second index gears 130A, 130B, the first and second index spools 131A, 131B rotate and operate to move a recess 132A, 132B, respectively, adjacent to or in juxtaposition with the manifold 114. As the blister strips 160A, 160B are advanced by the index spools 131A, 131B, the top sheets 166A, 166B of the blister strips 160A, 160B are peeled away from the bottom sheets 162A, 162B of the blister strips such that a pocket 164A, 164B thereof adjacent to the manifold 114 is opened and the powdered medicaments 168A, 168B therein is available for entrainment. More particularly, in order to expose the powdered medicaments 168A, 168B for each dose, the top sheets 166A, 166B are peeled away from the bottom sheets 162A, 162B, respectively, such that a pocket 164A, 164B in each bottom sheet 162A, 162B is opened or uncovered. Uncovering or opening of a pocket 164A, 164B is achieved by relative rotation between the index spools 131A, 131B and the top sheet take-up gears 150A, 150B. The index spools 131A, 131B essentially grip the bottom sheets 162A, 162B, respectively, and the top sheet take-up gears 150A, 150B, respectively, essentially grip the top sheets 166A, 166B. When the index spools 131A, 131B and the top sheet take-up gears 150A, 150B rotate relative to each other, the bottom sheets 162A, 162B and the top sheets 166A, 166B are peeled apart from each other. As will be described in more detail herein, the index spools 131A, 131B and the top sheet take-up gears 150A, 150B are driven via gearing to rotate in opposite directions, such that each top sheet 166A, 166B is peeled away from its respective bottom sheet 162A, 162B as the gears are driven. The dispensing subassembly 120 thus causes the respective leading pocket 164A, 164B to be opened and positioned into fluid communication with manifold 114 so that the powdered medicaments 168A, 168B of the opened pockets 164A, 164B are available for inhalation.

The first and second bottom sheet take-up gears 128A, 128B operate to wind up the empty bottom sheets 162A, 162B, respectively, of the blister strips 160A, 160B. As shown in FIG. 6, the first and second bottom sheet take-up gears 128A, 128B are attached to or integrally formed with spindles 129A, 129B, respectively. Via rotation of the first and second bottom sheet take-up gears 128A, 128B, the first and second spindles 129A, 129B rotate and operate to take-up the bottom sheets 162A, 162B as the inhaler device 100 is operated. The end of each bottom sheet 162A, 162B is anchored to the first and second bottom sheet take-up gears 128A, 128A such that progressive rotation of the first and second bottom sheet take-up gears 128A, 128B results in the bottom sheets 162A, 162B being wound therearound into a tight coil.

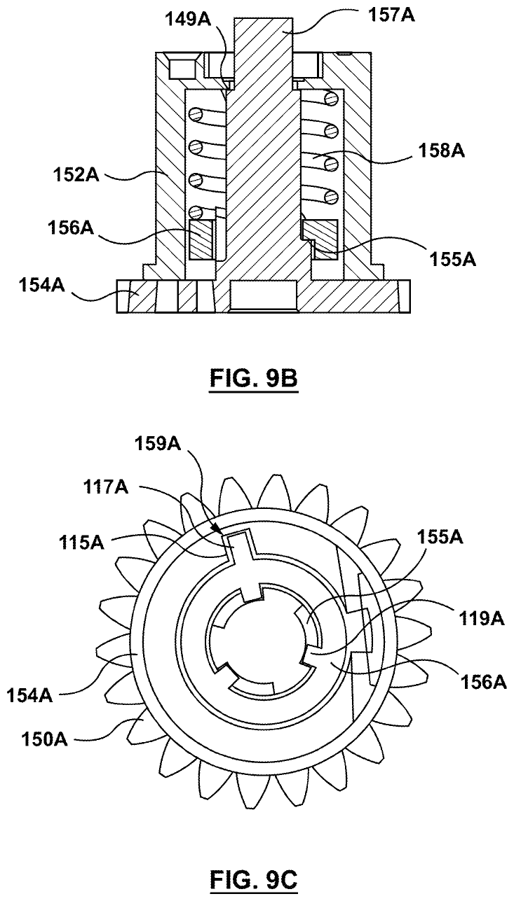

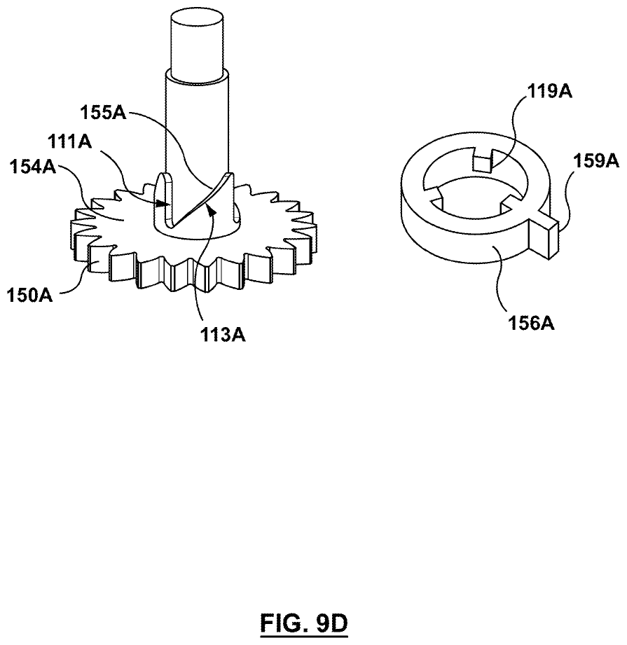

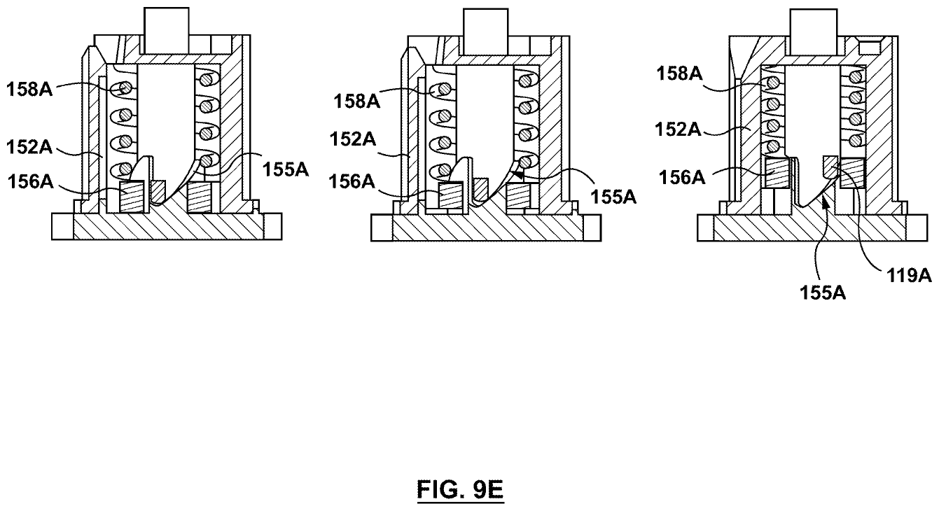

The first and second top sheet take-up gears 150A, 150B operate to wind up the top sheets 166A, 166B, respectively, of the blister strips 160A, 160B. As will be described in more detail herein with respect to FIGS. 9A-9E, the first and second top sheet take-up gears 150A, 150B are coupled to take-up hubs 152A, 152B, respectively. Via rotation of the first and second top sheet take-up gears 150A, 150B, the first and second take-up hubs 152A, 152B rotate and operate to take or wind up the top sheets 166A, 166B as the inhaler device 100 is operated. The end of each top sheet 166A, 166B is anchored to the first and second take-up hubs 152A, 152B such that progressive rotation of the first and second top sheet take-up gears 150A, 150B results in the top sheets 166A, 166B being wound therearound into a tight coil.

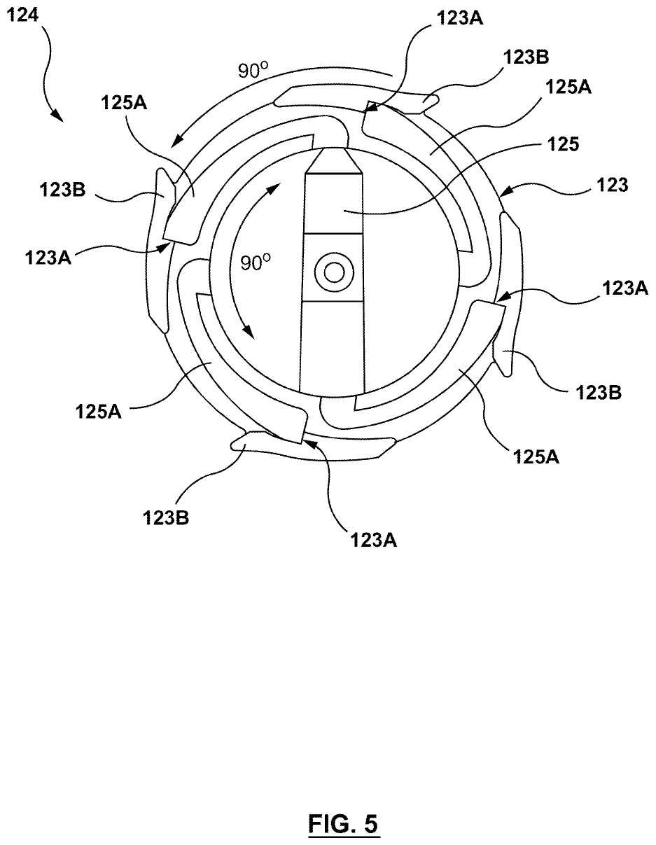

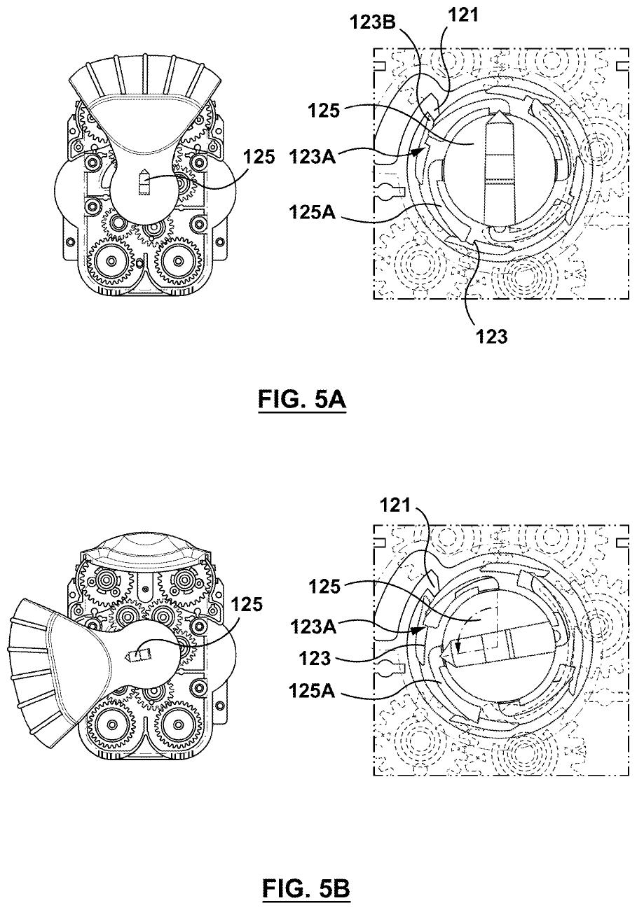

The central driver gear 122 of the dispensing subassembly 120 is attached to the mouthpiece cover 108 via the ratchet mechanism 124. The ratchet mechanism 124 is shown removed from the inhaler device 100 in FIG. 5. The ratchet mechanism 124 includes a ratchet 125 which is attached to the mouthpiece cover 108 and a ratchet gear 123 that is formed with or attached to the central driver gear 122. When the mouthpiece cover 108 is opened, the ratchet 125 is driven in the second opposing direction with the mouthpiece cover 108. The ratchet 125 in turn drives the ratchet gear 123 in the second opposing direction to advance or actuate the dispensing subassembly 120. When the mouthpiece cover 108 is returned to its closed position, the dispensing subassembly 120 is not advanced or actuated and remains stationary. An opening motion of the mouthpiece cover 108 is therefore transmitted to the central driver gear 122 but a closing motion of the mouthpiece cover 108 is not transmitted to the central driver gear 122.

More particularly, the ratchet gear 123 includes a plurality of circumferentially-spaced apart interior stop faces 123A and exterior stop faces 123B around an outer perimeter or edge thereof. The ratchet 125 includes a plurality of flexible ratchet arms 125A, which are configured to interact with the circumferentially-spaced apart interior stop faces 123A of the ratchet gear 123. The ratchet 125 rotates in a first direction with the mouthpiece cover 108 when the mouthpiece cover 108 is moved from a closed first position to an open second position. When rotating in the first direction, the ratchet arms 125A engage and drive the circumferentially-spaced apart interior stop faces 123A of the ratchet gear 123 as shown in FIG. 5B such that torque is transmitted to the central driver gear 122. Since the ratchet gear 123 is attached to or formed with the central driver gear 122, the central driver gear 122 rotates in the first direction concurrently with the mouthpiece cover 108. As described above, the movement of the mouthpiece cover 108 to the second position results in the opening and positioning of a pocket 164A, 164B of each blister strip 160A, 160B for subsequent simultaneous inhalation of the powdered medicaments 168A, 168B by the patient.

When the mouthpiece cover 108 is returned to its closed first position, however, the reverse rotation of the ratchet 125 is not transmitted to the central driver gear 122 because the ratchet arms 125A do not interact with the interior stop faces 123A of the ratchet gear 123. More particularly, when the ratchet 125 is rotated in the second opposing direction (i.e., counter clockwise), the ratchet arms 125A deflect radially inwards and no significant torque is transferred to the ratchet gear 123 and the central driver gear 122. Frictional drag between the ratchet 125 and ratchet gear 123 may tend to briefly drag the ratchet gear 123 in the second opposing direction (i.e., counter clockwise), but back-winding is prevented by interaction between one of the exterior stop faces 123B of the ratchet gear 123 and a detent arm 121 in a retainer plate of the inhaler device 100, as shown in FIG. 5A.

In an embodiment, a detent (not shown) may be disposed between an inner surface of the mouthpiece cover 108 and an outer surface the housing 102. The detent may be a mating protrusion and groove that is configured to temporarily resist or prevent the movement of the mouthpiece cover 108 relative to the housing 102 until a user applies a force to the mouthpiece cover 108 (i.e., when opening the mouthpiece cover 108) to release the detent by causing one of the mating features of the detent to exit or move beyond the other of the mating features of the detent. For example, a protrusion, bump or other raised structure may be formed on the outer surface of the housing 102 and a mating groove, dimple or other indentation structure may be formed on the inner surface of the mouthpiece cover 108. Alternatively, the protrusion, bump or other raised structure may be formed on the inner surface of the mouthpiece cover 108 and a mating groove, dimple or other indentation structure may be formed on the outer surface of the housing 102. The groove is configured to receive the indentation when the mouthpiece cover 108 is in the closed position of FIG. 1A. When a user applies a force sufficient to overcome the friction between the mating protrusion and groove, the mouthpiece cover 108 begins to open and move away from the closed position of FIG. 1A. The detent is configured to prevent or deter unintentional openings of the mouthpiece cover 108. In addition, the detent is configured to account for rotational clearances or tolerances within the ratchet mechanism 124 such that the dispensing and counter mechanisms in the inhaler device 100 are actuated upon the first or initial movement of the mouthpiece cover 108 away from the closed position towards the open position.

As best shown on FIG. 6, the central driver gear 122 directly or indirectly drives the remaining gears of the dispensing subassembly 120. This gear train arrangement provides for incremental indexing or advancement of the blister strips 160A, 160B via the first and second index gears 130A, 130B and also provides for winding of the top and bottom sheets of the blister strips 160A, 160B (via the top sheet take-up gears 150A, 150B and the bottom sheet take-up gears 128A, 128B, respectively) due to rotational movement in the first direction of the mouthpiece cover 108 from its closed first position to its open second position. As the central driver gear 122 rotates in the first direction with the mouthpiece cover 108, the central driver gear 122 mates with or directly drives the second index gear 130B to rotate in a second opposing direction. The second spool 131B, and the second blister strip 160B advanced thereby, thus also rotates in the second opposing direction. As the second index gear 130B rotates in the second opposing direction, the second index gear 130B mates with or directly drives the first index gear 130A to rotate in the first direction. The first spool 131A, and the first blister strip 160A advanced thereby, thus also rotates in the first direction.

In an embodiment, a first direction is clockwise, and a second opposing direction is counter-clockwise. In the depicted embodiment, the central driver gear 122 rotates in a clockwise direction when the mouthpiece cover 108 is opened. As such, the second index gear 130B (along with the second spool 131B and the second blister strip 160B) rotate in a counter-clockwise direction and the first index gear 130A (along with the first spool 131A and the first blister strip 160A) rotate in a clockwise direction. As will be appreciated by one of ordinary skill in the art, however, the first direction may alternatively be counter-clockwise and the second opposing direction may be clockwise so long as the gear train formed by the dispensing subassembly 120 causes the first blister strip 160A to move or advance in an opposite direction from the second blister strip 160B. In addition, as will be appreciated by one of ordinary skill in the art, the gear train formed by the dispensing subassembly 120 may include one or more idler gears (not shown) which alter the sequence of rotation between the central driver gear 122, the second index gear 130B and the first index gear 130A. The presence of such idler gears do not impact the overall function of the gear train, so long as the gear train causes the first blister strip 160A to move or advance in an opposite direction from the second blister strip 160B. For example, an idler gear (not shown) may be disposed between the central driver gear 122 and the first index gear 130A and the central driver gear 122 (rotating in the first direction) directly drives the idler gear to rotate in the second opposing direction. The idler gear may be positioned to directly drive the first index gear 130A in the first direction, and the first index gear 130A directly drives the second index gear 130B in the second opposing direction.

In order to keep tension applied to the blister strips 160A, 160B, the first and second bottom sheet take-up gears 128A, 128B rotate concurrently with and in the same direction as the first and second index gears 130A, 130B, respectively, and the first and second top sheet take-up gears 150A, 150B rotate concurrently with and in the opposite direction as the first and second index gears 130A, 130B, respectively. As the central driver gear 122 rotates in the first direction, the central driver gear 122 mates with or directly drives the first idler gear 126 to rotate in the second opposing direction and the first idler gear 126 mates with or directly drives the first bottom sheet take-up gear 128A to rotate in the first direction. The first spindle 129A, and the bottom sheet 162A wound thereby, thus also rotate in the first direction to wind or take-up the empty bottom sheet 162A of the first blister strip 160A as the first blister strip 160A is advanced by the dispensing subassembly 120. In addition, the first top sheet take-up gear 150A interacts with or is driven by the first index gear 130A. Since the first index gear 130A rotates in the first direction, the first top sheet take-up gear 150A is driven to rotate in the second opposing direction to wind up the top sheet 166A of the first blister strip 160A.

As the central driver gear 122 rotates in the first direction, the first idler gear 126 is driven to rotate in the second opposing direction as described above and the first idler gear 126 mates with or directly drives the second idler gear 127 to rotate in the first direction. The second idler gear 127 mates with or directly drives the second bottom sheet take-up gear 128B to rotate in the second direction. The second spindles 129B, and the bottom sheet 162B wound thereby, thus also rotate in the second direction to wind or take-up the empty bottom sheet 162B of the second blister strip 160B as the second blister strip 160B is advanced by the dispensing subassembly 120. In addition, the second top sheet take-up gear 150B interacts with or is driven by the second index gear 130B. Since the second index gear 130B rotates in the second opposing direction, the second top sheet take-up gear 150B is driven to rotate in the first direction to wind up the top sheet 166B of the second blister strip 160B.

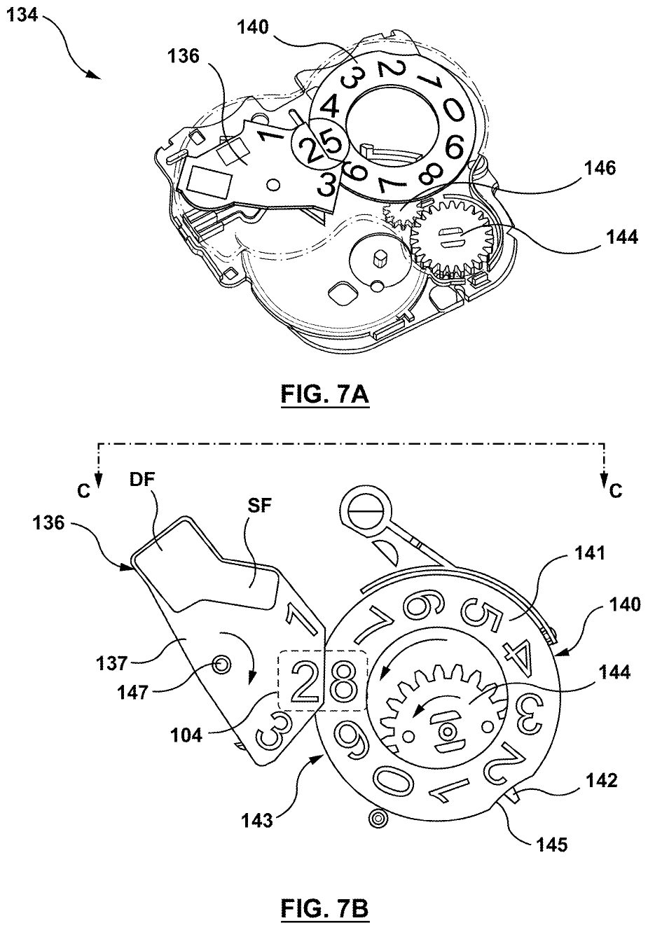

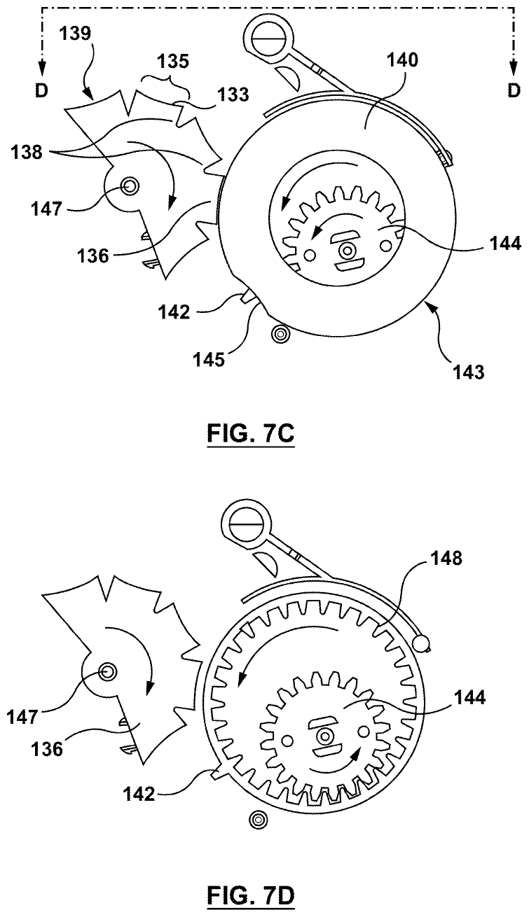

Turning now to FIGS. 7A-7D, the counter subassembly 134 will be described in more detail. The dispensing subassembly 120 advantageously directly drives the counter subassembly 134, so that the dose counter is incremented automatically and simultaneously with a medicament dose being indexed or delivered by the inhaler device 100. As such, beyond operating the mouthpiece cover 108 to actuate the dispensing subassembly 120, the user is not required to perform any additional operating steps to update the dose counter. The dose counter is incremented automatically when the mouthpiece cover 108 is opened, so it is intuitive to the user what the dose counter relates to.

The counter subassembly 134 includes a first count wheel or units ring 140 and a second count component or tens mechanism 136. The first count wheel 140 is driven from the dispensing subassembly 120 of the inhaler device 100 to rotate a fixed angle per dose, and in this embodiment is configured to display a second digit of a two-digit number of the available dose count. The second count component 136 is driven intermittently from the first count wheel 140 such that it rotates a fixed angle per revolution of the first count wheel 140, as will be described in more detail below. In the present embodiment, the second count component 136 displays a first digit of the two-digit number of the available dose count. As such, the first count wheel 140 and the second count component 136 collectively display a number of doses remaining within the inhaler device 100, as shown in FIGS. 7A and 7B. Although the embodiments of the counter subassemblies described herein display a two-digit number, it will be understood by one of ordinary skill in the art that the counting indicia may be revised to display a three-digit number if the total number of doses in the inhaler device exceed one-hundred.

A front or indicia-displaying face or surface 141 of the first count wheel 140 includes counting indicia disposed thereon that includes units or ‘ones’ digits. More particularly, as shown in FIGS. 7A and 7B, the counting indicia of the first count wheel 140 includes the digits 0, 1, 2, 3, 4, 5, 6, 7, 8, 9 in a circular pattern near an outer peripheral edge of the indicia-displaying surface 141. The angle between each digit is the same as the angle of rotation of the first count wheel 140 per dose. The indicia-displaying surface 141 is planar.

A front or indicia-displaying face or surface 137 of the second count component 136 includes counting indicia disposed thereon that includes ‘tens’ digits. The counting indicia of the second count component 136 includes the digits 3, 2, 1 and may also include a single flag SF and a double flag DF near an outer peripheral edge of the indicia-displaying surface 137. The angle between each counting indicia or digit is the angle of rotation of the second count component 136 for every revolution of the first count wheel 140. The indicia-displaying surface 137 is planar.

The display window 104 in the housing 102 is positioned such that one digit on the first count wheel 140 and one digit on the second count component 136 are visible within the display window, and adjacent to each other to form a two-digit number. The displayed digit on the second count component 136 reflects or tracks the number of ‘tens’ of doses remaining in the inhaler device 100, while the displayed digit on the first count wheel 140 reflects or tracks the number of ‘ones’ of doses remaining. When the number of remaining doses is below ten, the second count component 136 will display the single flag SF in the display window 104 instead of or in place of a zero digit. In an embodiment, for example, the single flag SF may be a colored block with no digits thereon to indicate to the user that they are nearing the end of available doses within the inhaler device 100. When the number of remaining doses reaches zero, the second count component 136 will display the double flag DF in the display window 104 instead of a digit. In an embodiment, for example, the double flag DF may be a colored block with no digits thereon that covers the digit of the first count wheel 140 to give clear visual feedback to the user that no doses remain within the inhaler device 100. The double flag DF is configured to cover the digit of the first count wheel 140 when no doses remain within the inhaler device 100.

In the embodiment of FIGS. 7A-7D, the units or ‘ones’ digits are circumferentially disposed on the first count wheel 140 in a descending order in a first direction, and the ‘tens’ digits are disposed near a perimeter of the second count component 136 in a descending order in a second opposing direction. While the counter subassemblies described herein display a number of doses remaining within the inhaler device 100, it will be understood by one of ordinary skill in the art that the counter subassemblies may be modified to display a number of doses delivered by the inhaler device by reversing the order of the counting indicia disposed on the counting subassembly.

Turning now to FIGS. 7C and 7D, the structure and operation of the first count wheel 140 and the second count component 136 will be described in more detail. FIG. 7C is a sectional view taken along line C-C of FIG. 7B, which is along an intermediate position between the indicia-displaying surfaces and the opposing back surfaces of the count subassembly. FIG. 7D is a sectional view taken along line D-D of FIG. 7C, which is adjacent to the opposing back surfaces of the count subassembly. The relative terms “front” and “back” are utilized herein for sake of illustration only and relate to how an inhaler device is customarily positioned by a user during use with a front of the inhaler device including the display window for the counter mechanism.

The first count wheel 140 is an annular or ring-shaped component having the front or indicia-displaying surface 141 and an opposing back surface which includes a counter gear 148, as best shown in the sectional view of FIG. 7D. The counter gear 148 may be integrally formed on or attached to the first count wheel 140. An outer circumferential surface or side 143 extends between the front and back surfaces of the first count wheel 140. The outer circumferential side 143 may be stepped, with a smaller diameter along the back surface of the first count wheel 140 and a larger diameter along the front surface of the first count wheel 140. The stepped nature of the outer circumferential side 143 is apparent via a comparison of the relative diameters thereof in FIGS. 7C and 7D. Stated another way, the indicia-displaying surface 141 of the first count wheel 140 has a greater outer diameter than the counter gear 148.

Adjacent to the counter gear 148, the first count wheel 140 includes a single tooth or protrusion 142 extending radially outward from the outer circumferential side 143. Along the front or indicia-displaying surface 141 of the first count wheel 140, the outer circumferential side 143 is generally circular with a clearance cutout or indent 145 formed around the single tooth 142. The function of the clearance indent 145 will be described in more detail below.

The second count component 136 is configured to rotate around a pivot point 147. The second count component 136 is a non-annular or partial-disk component having the front or indicia-displaying surface 137 and an opposing back surface which includes a plurality of notches 138, as shown in the sectional views of FIGS. 7C and 7D. An outer surface or side 139 extends between the front and back surfaces of the second count component 136. The outer side 139 may be stepped, with a smaller radial dimension along the back surface of the second count component 136 and a larger radial dimension along the front surface of the second count component. The stepped nature of the outer side 139 is apparent via a comparison of the relative diameters thereof in FIGS. 7B and 7C.

The plurality of notches 138 are formed in the outer side 139 of the second count component 136 and do not extend through the front or indicia-displaying surface 137. In an embodiment, the second count component 136 includes four notches 138 but the number of notches is exemplary and depends on the capacity or total number of available doses within the inhaler device 100. Each notch 138 extends radially inward toward the pivot point 147 of the second count component 136 and is configured to mate with or receive the single tooth 142 of the first count wheel 140. The outer side 139 of the second count component 136 may be considered to include a plurality of segments 135, with each segment 135 extending between two neighboring or adjacent notches 138. Along each segment 135, the outer side 139 of the second count component 136 is concave and forms an inverted curved or arc-shaped indent 133 that is coincident with the outer circumferential side 143 of the first count wheel 140.

The first count wheel 140 is driven by the second bottom sheet take-up gear 128B of the dispensing subassembly 120 such that the first count wheel 140 rotates a fixed angle every time a dose is dispensed. More specifically, a transfer gear 144 is attached to an opposing end of the second spindle 129B of the second bottom sheet take-up gear 128B so that the transfer gear 144, the second spindle 129B, and the second bottom sheet take-up gear 128B rotate simultaneously as an assembly. When the mouthpiece cover 108 is opened, the transfer gear 144 thus rotates in the second opposing direction with the second bottom sheet take-up gear 128B. The transfer gear 144 mates with or directly drives an idler gear 146 to rotate in the first direction, and the idler gear 146 mates with or directly drives the counter gear 148 to rotate in the second opposing direction. Thus, the counter gear 148 rotates in the same direction as the transfer gear 144. As will be apparent to those of ordinary skill in the art, rotation of the transfer gear 144 and the counter gear 148 in the same direction may also be accomplished via a gear train in which the transfer gear 144 directly drives the counter gear 148 in the same direction. For example, as depicted in FIGS. 7B-7D, the desired rotation scheme is accomplished when the counter gear 148 is an internal gear and the transfer gear 144 is a spur or planetary gear so that the transfer gear 144 meshes with or directly drives the internal counter gear 148 in the same direction. The first count wheel 140 is attached to the counter gear 148 to rotate therewith as an assembly, and thus the first count wheel 140 rotates in the second opposing direction when the mouthpiece cover 108 is opened. Thus, various suitable gear train may be utilized herein such that the transfer gear 144 and the counter gear 148 rotate in the same direction.

As best shown on FIG. 7C, the second count component 136 is positioned adjacent or next to the outer circumferential side 143 of the first count wheel 140, such that the single tooth 142 of the first count wheel 140 engages a notch 138 of the plurality of notches 138 of the second count component 136 once per revolution of the first count wheel 140 to intermittently rotate the second count component 136. Stated another way, each time that the first count wheel 140 makes a complete revolution, the single tooth 142 engages a notch 138 of the second count component 136 and turns or rotates the second count component 136 a fixed amount. Thus, there is a fixed rotation of the second count component 136 once per revolution of the first count wheel 140. Since the first count wheel 140 directly drives the second count component 136, the second count component 136 rotates in an opposing direction than the first count wheel 140. In the embodiment of FIGS. 7A-7D, the second count component 136 is configured to rotate in the first direction and the first count wheel 140 is configured to rotate in the second opposing direction. The second count component 136 is stationary and does not rotate when the single tooth 142 of the first count wheel 140 is not engaged a notch 138 of the second count component 136.

The second count component 136 is disposed next to or side-by-side in a common plane with the first count wheel 140, and thus the second count component 136 rotates on a different axis of rotation than the first count wheel 140. Stated another way, the first count wheel 140 rotates about a first axis and the second count component 136 rotates about a second axis, with the second axis being parallel to and spaced apart from the first axis.

As stated above, along each segment 135, the outer side 139 of the second count component 136 is concave and forms the arc-shaped indent 133 that is coincident with the outer circumferential side 143 of the first count wheel 140. As best shown on FIG. 7C, the geometry of the arc-shaped indents 133 is coincident with the circular profile of the first count wheel 140 to prevent the second count component 136 from inadvertently rotating when not engaged with the first count wheel 140. Particular, this geometry or profile of the outer side 139 of the second count component 136 ensures that the second count component 136 does not rotate, and remains stationary, when the single tooth 142 of the first count wheel 140 is not engaged with one of the notches 138 of the second count component 136. Conversely, when the single tooth 142 of the first count wheel 140 is engaged with or received within one of the notches 138 of the second count component 136, the clearance indent 145 of the first count wheel 140 allows the second count component 136 to briefly rotate with the first count wheel 140 to change the tens display.

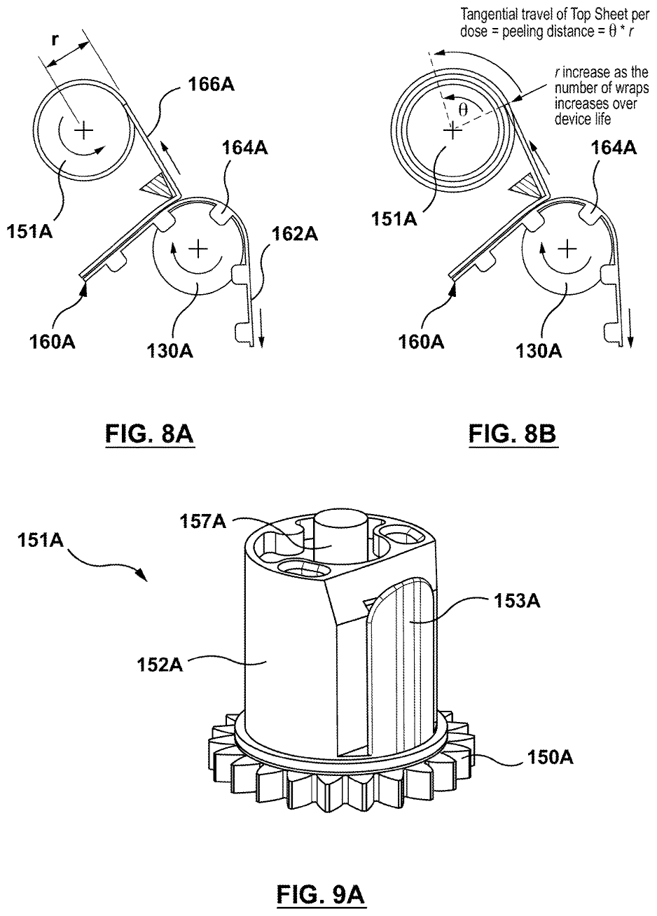

Turning now to FIGS. 8A and 8B and 9A-9E, first and second tensioning mechanisms 151A, 151B will be described in more detail. The tensioning mechanisms 151A, 151B function to peel the top sheets 166A, 166B from the first and second blister strips 160A, 160B, respectively, in a manner that maintains consistent peeling distance or amount over the device lifetime. More particularly, the tensioning mechanisms 151A, 151B ensure that the peeling distances of the top sheets 166A, 166B are configured to properly open the pockets 164A, 164B for each dose in order to achieve effective dispensing of the powdered medicaments 168A, 168B to the user. A pocket may not be fully exposed if the peeling distance or amount is too low, which makes it more difficult to achieve sufficient evacuation of the powdered medicament disposed therein upon inhalation. Further, if the peeling distance or amount is too high, the next or following pocket may be prematurely exposed and there is a risk of some medicament disposed therein being lost, which could result in an under dose on the next dispense. The tensioning mechanisms 151A, 151B also function to maintain sheet tension of the top sheets 166A, 166B over the device lifetime. The top sheets 166A, 166B need to be under consistent tension to ensure proper operation of the inhaler device 100. The tension of each top sheet 166A, 166B is linked to the force required by the user to operate the inhaler device 100 and move the mouthpiece cover 108. Ensuring consistent tension in the top sheets 166A, 166B thus provides a more consistent user experience over the device lifetime. Maintaining consistent tension in the top sheets 166A, 166B also results in more consistent and lower peak mechanical stress in the top sheets 166A, 166B and the surrounding components, which reduces the risk of mechanical failure during use.

The first tensioning mechanism 151A is associated with the first top sheet take-up gear 150A, for winding up the top sheet 166A of the first blister strip 160A, and the second tensioning mechanism 151B is associated with a second top sheet take-up gear 150B (see FIGS. 4A and 4B), for winding up the top sheet 166B of the second blister strip 160B. Only first tensioning mechanism 151A is described herein for sake of brevity, as the second tensioning mechanism 151B operates in the same manner. It will be apparent to those of ordinary skill in the art that certain features or components of the second tensioning mechanism 151B (i.e., the cam surface described herein) will be modified to extend in an opposing direction from the description below such that the second tensioning mechanism 151B is configured to operate in an opposite direction than the first tensioning mechanism 151A. Advantageously, the first tensioning mechanism 151A and the second tensioning mechanism 151B use some components of identical design (i.e., the compression spring and the take-up hub described herein), which may reduce the cost of manufacturing and assembly compared to other inhalation devices in which all components need to be manufactured individually for each side of the device in order to operate in opposite directions.

The tensioning mechanism 151A includes the first top sheet take-up gear 150A, the take-up hub 152A having a hook 153A integrally formed thereon or fixed thereto, a base 154A having a cam surface 155A integrally formed thereon or fixed thereto, a nut 156A, a shaft 157A, and a compression spring 158A that extends or is disposed between the nut 156A and a top end of the take-up hub 152A. The compression spring 158A biases the nut 156A downwards, towards the base 154A, into the cam surface 155A. The compression spring 158A is disposed about or around the shaft 157A and is longitudinally or axially adjacent to the nut 156A, and the take-up hub 152A is disposed about or around the nut 156A and the compression spring 158A. Stated another way, the take-up hub 152A encircles or surrounds the nut 156A and the compression spring 158A which are contained therein.

The top sheet 166A of the first blister strip 160A is secured or attached to the take-up hub 152A via the hook 153A such that as the take-up hub 152A rotates, the top sheet 166A of the first blister strip 160A wraps around it. The hook 153A is configured to attach to a leading end of the top sheet 166A such that rotation of the take-up hub 152A results in winding or wrapping of the top sheet 166A around the take-up hub 152A. Since the take-up hub 152A completes multiple rotations over the lifetime of the inhaler device 100, the top sheet 166A of the first blister strip 160A wraps over itself multiple times, causing its radial position on the take-up hub 152A to increase, i.e., a radial distance of each subsequent winding of the top sheet 166A from the take-up hub 152A increases with each winding. Since rotation of the base 154A corresponds to the rotation of the first top sheet take-up gear 150A, the base 154A rotates a fixed amount for each dose and the distance that the top sheet 166A of the first blister strip 160A is peeled relative to the bottom sheet 162A is determined by the tangential travel of the top sheet 166A of the first blister strip 160A at the take-up hub 152A. This tangential travel may be calculated as θ*r, where θ is the angle of rotation of the take-up hub 152A per dose, and r is the radial position of the top sheet 166A of the first blister strip 160A on the take-up hub 152A. As the radial position of the top sheet 166A of the first blister strip 160A increases, this tangential distance therefore increases for a fixed rotation of the take-up hub 152A and, without any modification of the mechanism, the top sheet 166A of the first blister strip 160A would be peeled further relative to the bottom sheet 162A later in the device life. This is known as the wrapping effect and is illustrated via a comparison of FIG. 8A and FIG. 8B. FIG. 8A is a schematic view of the first blister strip 160A early in the device life, and FIG. 8B is a schematic view of the first blister strip 160A late in the device life. FIG. 8B illustrates how the effective diameter of the take-up hub 152A increases as the first blister strip 160A is wrapped around the take-up hub 152A.

In order to ensure that the top sheet 166A of the first blister strip 160A is peeled the same amount each dose and compensate for the wrapping effect, the tensioning mechanism 151A includes the cam surface 155A, the nut 156A, and the compression spring 158A. The function of the cam surface 155A, the nut 156A, and the compression spring 158A is to provide a constant driving tension to the top sheet 166A over the entire strip length. As will be described in more detail herein, increased tension along the top sheet 166A results in rotation of the take-up hub 152A relative to the base 154A to reduce the tension along the top sheet 166A. When the take-up hub 152A rotates relative to the base, the compression spring 158A is compressed and axial compression of the compression spring 158A is transferred to torque applied to the take-up hub 152A.

With reference to FIGS. 9B-9D, the structure of the tensioning mechanism 151A will be described in more detail. The base 154A is attached to or formed integrally with the first top sheet take-up gear 150A to rotate as an assembly when the first top sheet take-up gear 150A is rotationally driven. For example, the base 154A may include a plurality of gear teeth integrally formed with or fixed to an outer circumferential surface thereof to form the first top sheet take-up gear 150A. The shaft 157A extends from the base 154A and is attached to or formed integrally with the base 154A to rotate therewith. Thus, the shaft 157A, the base 154A, and the first top sheet take-up gear 150A rotate as an assembly when the first top sheet take-up gear 150A is rotationally driven.

The nut 156A is disposed between and coupled to each of the take-up hub 152A and the base 154A. The nut 156A is disposed about or around the shaft 157A and is coupled to the base 154A via at least one inwardly-extending rib 119A that projects or extends radially inwards from an inner circumferential surface of the nut 156A. In an embodiment, the nut 156A includes a plurality of inwardly-extending ribs 119A. In an embodiment, the plurality of inwardly-extending ribs 119A are circumferentially spaced apart in equal increments. More particularly, as best shown on FIG. 9C, the inwardly-extending ribs 119A of the nut 156A are disposed onto and engage the cam surface 155A of the base 154A. Further, the nut 156A is coupled to the take-up hub 152A via a splined coupling 159A such that the take-up hub 152A rotates with the nut 156A and no relative rotation is permitted therebetween. Stated another way, due to the splined coupling 159A, the take-up hub 152A is rotationally locked to the nut 156A such that the nut 156A and the take-up hub 152A rotate as an assembly. As best shown on FIG. 9C, the splined coupling 159A includes an outwardly-extending rib 117A which projects or extends radially outwards from an outer circumferential surface of the nut 156A and is received within an axial slot 115A of the take-up hub 152A. The outwardly-extending rib 117A is permitted to slide or move in an axial direction along the axial slot 115A such that the nut 156A is permitted to slide or move in an axial direction relative to the take-up hub 152A, but the outwardly-extending rib 117A does not permit the nut 156A to rotate relative to the hub 152A.

When the base 154A rotates in the second opposing direction with the first top sheet take-up gear 150A, the nut 156A and the take-up hub 152A also rotate in the second opposing direction due to the interaction between the nut 156A with the compression spring 158A and the cam surface 155A of the base 154A. More particularly, as the base 154A is being rotationally driven in the second opposing direction, the take-up hub 152A is configured to rotate in the second opposing direction via engagement of the inwardly-extending ribs 119A of the nut 156A with the cam surface 155A. As shown on FIG. 9D, the cam surface 155A includes alternating sections of vertical surfaces 111A and angled or inclined surfaces 113A. Vertical surfaces 111A extend generally parallel to a longitudinal axis of the shaft 157A. As the base 154A is being rotationally driven in the second opposing direction (i.e., the counter-clockwise direction in this embodiment), the nut 156A and the take-up hub 152A rotationally locked therewith rotate with the base 154A in the second opposing direction. Interaction between the compression spring 158A, the nut 156A and the cam surface 155A result in a torque being applied from the cam surface 155A to the nut 156A, in the direction that would push the nut 156A down the cam surface 155A. The compression spring 158A is pushing the nut 156A against the inclined surfaces 113A of the cam surface 155A, which results in torque acting on the take-up hub 152A that drives the take-up hub 152A in the second opposing direction. As a result of this interaction when the base 154A moves counter-clockwise, the nut 156A and the take-up hub 152A also rotate counter-clockwise with the base 154A. It will be understood by one of ordinary skill in the art that the pattern of the cam surface 155A is exemplary. A thread or other ramped surface may be utilized as the cam surface 155A.

Although the take-up hub 152A rotates in the second opposing direction with the base 154A due to the nut 156A interacting with the compression spring 158A and the cam surface 155A of the base 154A, relative rotation between the take-up hub 152A and the base 154A is permitted. More particularly, when sufficient torque is applied between the take-up hub 152A and the base 154A, the take-up hub 152A will rotate relative to the base 154A. Since the take-up hub 152A is permitted to rotate relative to the base 154A in a direction that will reduce tension in the top sheet 166A (i.e., in the first direction), this relative rotation stabilizes or balances the tension in the top sheet 166A via deflection of the compression spring 158A. The tensioning mechanism 151A is therefore acting as a torsion or torque limiter between the take-up hub 152A and the base 154A to control the tension in the top sheet 166A of the first blister strip 160A.

In addition to the torque being applied from the cam surface 155A to the nut 156A, there is also a torque in the opposing direction acting on the nut 156A from its interaction with the hub 152A via the outwardly-extending rib 117A. This opposing torque comes from the tension in the top sheet 166A, which is acting to apply torque to the hub 152A. The two opposing torques on the nut 156A are in equilibrium, and as such the compression spring 158A (via the nut 156A and cam surface 155A) effectively balances the tension in the top sheet 166A. If the tension in the top sheet 166A increases, then the nut 156A will move further up the cam surface 155A and the spring force of the compression spring 158A will increase to compensate for the increased tension.

More particularly, due to the wrapping effect described above, as the take-up hub 152A rotates, the radial position of the top sheet 166A of the first blister strip 160A increases. As a result of this increased radial position, the take-up hub 152A attempts to peel a longer length of top sheet 166A and the tension on the top sheet 166A increases due to a change in the peeling angle between the top sheet 166A of the first blister strip 160A and the bottom sheet 162A of the first blister strip 160A. When tension on the top sheet 166A increases, the torque that the top sheet 166A applies to the take-up hub 152A increases as well. Rather than the torque continuing to increase, the take-up hub 152A will start to rotate relative to the base 154A in the second direction, and the nut 156A will move helically up the cam surface 155A of the base 154A, thereby compressing the compression spring 158A. More particularly, when the take-up hub 152A and the nut 156A rotationally locked thereto begin to rotate in the second direction due to the increased tension on the top sheet 166A, the base 154A and the cam surface 155A remain stationary and the inwardly-extending ribs 119A of the nut 156A move along the inclined surfaces 113A of the cam surface 155A in a direction towards the compression spring 158A, i.e., higher up the cam surface 155A. The compression spring 158A compresses as the nut 156A presses against it. As the nut 156A moves relative to the base 154A, the nut 156A also moves axially relative to the take-up hub 152A because the outwardly-extending rib 117A of the nut 156A is permitted to move axially within the axial slot 115A of the take-up hub 152A. Thus, rotation of the take-up hub 152A relative to the base 154A results in axial movement of the nut 156A relative to the take-up hub 152A and relative to the base 154A, and further axial movement of the nut 156A towards the compression spring 158A axially compresses the compression spring 158A. Since the take-up hub 152A is rotationally locked to the nut 156A via the splined coupling 159A, the nut 156A transfers the axial force of the compressed spring 158A into torque on the take-up hub 152A. The combination of the compression spring 158A, the nut 156A, and the cam surface 155A is thereby providing torque to react or counteract the relative rotation between the take-up hub 152A and the base 154A, which is acting to maintain consistent tension in the top sheet 166A.

The take-up hub 152A is axially constrained relative to the base 154A via a clip or retention feature 149A disposed between the shaft 157A and the take-up hub 152A. More particularly, since the compression spring 158A is acting to axially separate the take-up hub 152A and the base 154A, the retention feature 149A (best shown on FIG. 9B) is disposed between the top end of the take-up hub 152A and the compression spring 158A to maintain the correct relative axial position between the take-up hub 152A and the base 154A. The retention feature 149A may be a clip, a bayonet, or other component suitable to maintain the correct relative axial position between the take-up hub 152A and the base 154A. In another embodiment (not shown), the retention feature may be attached to the interior of the housing 102 to maintain the correct relative axial position between the take-up hub 152A and the base 154A.

FIG. 9E illustrates how the position of the nut 156A changes over the lifetime of the device. In its initial assembled state shown in the left image, prior to attachment of the take-up hub 152A to the first blister strip 160A, the nut 156A rests at the bottom end of the cam surface 155A. The cam surface 155A is designed such that, in this position, there is no resultant torque between the take-up hub 152A and the base 154A, even though there may be axial force from the compression spring 158A. When the device is assembled, the top sheet 166A is assembled under some tension to ensure that peeling is effective from the first dose and as a result, the nut 156A is lifted up the cam surface 155A slightly away from the vertical surfaces 111A of the cam surface 155A. More particularly, as shown in the middle image, when the device is assembled the top sheet 166A is attached to the take-up hub 152A, the take-up hub 152A is rotated relative to the base 154A, and the nut 156A moves up the cam surface 155A. The compression spring 158A is deflected or slightly compressed from its uncompressed length so that the compression spring 158A has a preload force. The preload force ensures that the tension in the top sheet 166A of the first blister strip 160A is sufficiently high to peel it away from the bottom sheet 162A of the first blister strip 160A at the start of the device life. The specifications of the compression spring 158A and the angle of the cam surface 155A should be configured to provide a minimum tension in the top sheet 166A of the first blister strip 160A that is higher than the maximum force required to peel the top sheet 166A of the first blister strip 160A from the bottom sheet 162A of the first blister strip 160A. In this assembled state, the combination of the compression spring 158A, the nut 156A, and the cam surface 155A provide a torque between the base 154A and the take-up hub 152A, which is reacted by tension in the top sheet 166A. Over the lifetime of the inhaler device 100 as shown in the right image, the tension in the top sheet 166A of the first blister strip 160A increases. Due to the increased tension of the top sheet 166A, the nut 156A moves further up the cam surface 155A so that the increased tension of the top sheet 166A is balanced by further deflection of the compression spring 158A.

The angle or slope of each inclined surface 113A of the cam surface 155A is configured to maintain consistent sheet tension of the top sheets 166A, 166B over the device lifetime. As described above, the top sheets 166A, 166B need to be under consistent tension to ensure proper operation of the inhaler device 100. In general, the angle or slope of each inclined surface 113A of the cam surface 155A is selected to ensure that the nut 156A moves along the cam surface 155A of the base 154A during operation of the inhaler device 100 and does not move beyond or past the vertical surfaces 111A. In an embodiment, the inclined surfaces 113A of the cam surface 155A extend at an angle between 35 degrees and 55 degrees relative to the longitudinal axis of the base 154A. In an embodiment, the inclined surfaces 113A of the cam surface 155A extend at an angle between 40 degrees and 50 degrees relative to the longitudinal axis of the base 154A. In an embodiment, the inclined surfaces 113A of the cam surface 155A extend at an angle of approximately 45 degrees relative to the longitudinal axis of the base 154A, with approximately as used herein including a tolerance of three degrees. In an embodiment, the inclined surfaces 113A of the cam surface 155A have a slope between 0.70 and 1.0. In an embodiment, the inclined surfaces 113A of the cam surface 155A have a slope between 0.80 and 0.95. In another embodiment, the inclined surfaces 113A of the cam surface 155A have a slope between 0.7 and 1.4. In another embodiment, the inclined surfaces 113A of the cam surface 155A have a slope between 1.0 and 1.4. The slope of the inclined surfaces 113A of the cam surface 155A may be constant over the length of the inclined surface, or may vary over the length of the inclined surface.

The radial width of the inclined surface 113A of the cam surface 155A is configured to optimize the amount of friction between the nut 156A and the cam surface 155A. In general, larger dimensions of the radial width of the inclined surface 113A of the cam surface 155A result in increased friction between the components while smaller dimensions of the radial width of the inclined surface 113A of the cam surface 155A may result in the nut 156A undesirably falling off the cam surface 155A. In an embodiment, each inclined surface 113A of the cam surface 155A has a radial width between 1 mm and 3 mm. In an embodiment, each inclined surface 113A of the cam surface 155A has a radial width between 1.5 mm and 2.5 mm. In an embodiment, each inclined surface 113A of the cam surface 155A has a radial width of approximately 2 mm, with approximately as used herein including a tolerance of 0.2 mm.

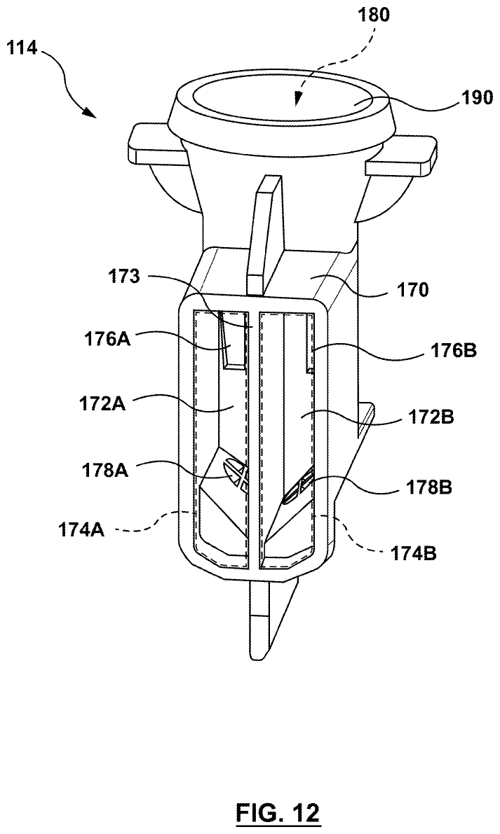

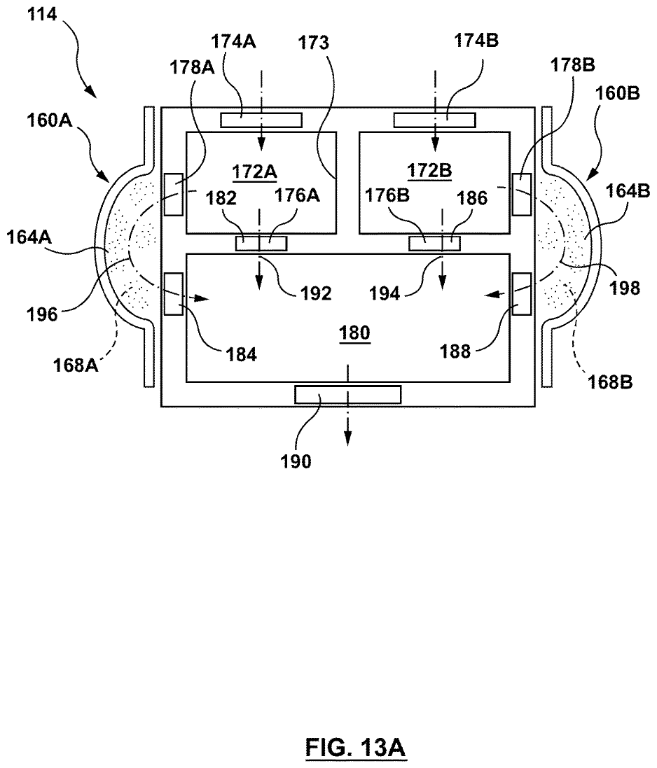

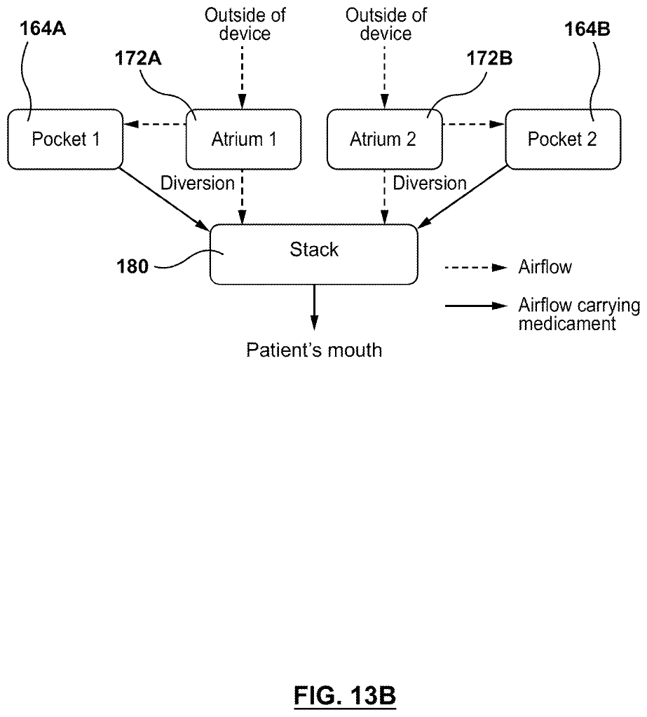

With reference to FIGS. 10-14, the manifold 114 will be described in more detail. FIGS. 10 and 12 are perspective views of the manifold 114 removed from the inhaler device 100 for sake of illustration only. FIG. 10A is a sectional view taken along line A-A of FIG. 10, and FIG. 11 illustrates a relative positioning between the manifold 114 and the inlet vents 106 of the housing 102. FIG. 13A is a schematic view illustrating an airflow path through the manifold 114, and FIG. 13B is a schematic flowchart illustrating the airflow path through the manifold 114.

The manifold 114 is configured for simultaneous delivery of powdered medicament 168A, 168B from respective open blister pockets 164A, 164B, of each of the first blister strip 160A and the second blister strip 160B, respectively. The manifold 114 includes a body 170 that defines a first space or atrium 172A, a second space or atrium 172B, and a stack 180. As will be explained in more detail herein, the separated distinct compartments or spaces of the first atrium 172A, the second atrium 172B, and the stack 180 split up, divide, or otherwise separate an inhalation airflow, which is drawn into the manifold 114 by a user, into multiple airflow paths through the body 170 of the manifold 114. More specifically, when an inhalation force is applied through the central opening 112 of the mouthpiece 110, an inhalation airflow is drawn into the first and second atriums 172A, 172B of the manifold 114 via the inlet vents 106 of the inhaler device 100. The first and second atriums 172A, 172B are disposed adjacent to, or are in a juxtaposed relation with, the inlet vents 106. Once the inhalation airflow enters into the manifold 114, it splits or divides into four airflow paths as it travels through the body 170 of the manifold, namely, a first diversion airflow path 192, a second diversion airflow path 194, a first entrainment airflow path 196, and a second entrainment airflow path 198. Stated another way, each of the first diversion airflow path 192, the second diversion airflow path 194, the first entrainment airflow path 196, and the second entrainment airflow path 198 are respective airflow portions of the inhalation airflow which is drawn into the manifold 114.

In this embodiment, the first atrium 172A and the second atrium 172B are disposed laterally adjacent to each other, or side-by-side, on a single side of the body 170 of the manifold 114. The first atrium 172A and the second atrium 172B are separated from each other by a divider wall 173 such that the first atrium 172A is not in fluid communication with the second atrium 172B. The first atrium 172A includes a single atrium inlet 174A, and the second atrium 172B includes a single atrium inlet 174B. The atrium inlets 174A, 174B are separate from each other and may also be considered the inlets of the manifold 114. As such, the manifold 114 includes two inlets, the atrium inlet 174A leading or entering into the first atrium 172A and the atrium inlet 174B leading or entering into the second atrium 172B.

The first atrium 172A includes a first atrium outlet 176A and a second atrium outlet 178A, and the second atrium 172B includes a first atrium outlet 176B and a second atrium outlet 178B. As will be explained in more detail herein, the first atrium outlet 176A, 176B of each of the first and second atriums 172A, 172B, respectively, directs or guides flow directly into the stack 180 and the second atrium outlet 178A, 178B of each of the first and second atriums 172A, 172B, respectively, directs or guides flow into an open pocket 164A, 164B, respectively, of the first and second blister strips 160A, 160B, respectively. In an embodiment, the profile or shape of the first atrium outlet 176A, 176B is substantially rectangular or oblong. However, the profile or shape of the first atrium outlets 176A, 176B is not limited to the shape depicted herein and may alternatively be circular, triangular, or any other shape deemed suitable for the purposes described herein. Similarly, the profile or shape of the second atrium outlets 178A, 178B is substantially circular and includes a grill or cross-piece 197 (see FIG. 10) spanning thereover to encourage increased turbulence in the airflow. However, the profile or shape of the second atrium outlets 178A, 178B is not limited to the shape depicted herein and may alternatively be rectangular, oblong, oval, triangular, or any other shape deemed suitable for the purposes described herein and may or may not include a grill spanning thereover.

As best shown on FIG. 13A, the stack 180 is in fluid communication with each of the first atrium 172A, the second atrium 172B, the open pocket 164A of the first blister strip 160A, and the second open pocket 164B of the second blister strip 160B. The stack 180 has four inlets, namely, a first stack inlet 182, a second stack inlet 184, a third stack inlet 186, and a fourth stack inlet 188. The first stack inlet 182 is aligned with the first atrium outlet 176A of the first atrium 172A, such that the stack 180 and the first atrium 172A are in fluid communication with each other. The second stack inlet 184 is aligned with the open pocket 164A of the first blister strip 160A, such that the second stack inlet 184 is further in fluid communication with the second atrium outlet 178A of the first atrium 172A via the open pocket 164A. The third stack inlet 186 is in fluid communication with the second atrium outlet 176B of the second atrium 172B, such that the stack 180 and the second atrium 172B are in fluid communication with each other. The fourth stack inlet 188 is in fluid communication with the open pocket 164B of the second blister strip 160B, such that the fourth stack inlet 188 is further in fluid communication with the second atrium outlet 178B of the second atrium 172B via the open pocket 164B.

The profile or shape of each of the first stack inlet 182 and the third stack inlet 186 is substantially rectangular or oblong. However, the profile or shape of the first and third stack inlets 182, 186 is not limited to the shape depicted herein and may alternatively be circular, triangular, or any other shape deemed suitable for the purposes described herein. Similarly, the profile or shape of the second stack inlet 184 and the fourth stack inlet 188 is substantially circular and includes a grill or cross-piece 195 (see FIG. 10) spanning thereover to encourage increased turbulence in the airflow. However, the profile or shape of the second and fourth stack inlets 184, 188 is not limited to the shape depicted herein and may alternatively be rectangular, oblong, oval, triangular, or any other shape deemed suitable for the purposes described herein and may or may not include a grill spanning thereover.

The stack 180 is a single stack with a single stack outlet 190. Thus in the present embodiment, the stack 180 has only one stack outlet 190. The stack outlet 190 may also be considered the outlet of the manifold 114. As such, the manifold 114 includes only one outlet. A shape or profile of the stack outlet 190 is oval. However, the profile or shape of the stack outlet 190 is not limited to the shape depicted herein and may alternatively be circular, rectangular, oblong, triangular, or any other shape deemed suitable for the purposes described herein. When the manifold 114 is assembled into the inhaler device 100, the stack outlet 190 is aligned and in fluid communication with the central opening 112 of the mouthpiece 110.

The second atrium outlet 178A of the first atrium 172A is in fluid communication with the second stack inlet 184 to define the first entrainment airflow path 196 associated with an open blister pocket 164A of the first blister strip 160A. As the airstream flows through the open blister pocket 164A, it picks up the powdered medicament 168A disposed within the open blister pocket 164A. Thus, by passing through the open blister pocket 164A, the powdered medicament 168A is drawn in and transported by the airstream into the stack 180. After entrainment, the airstream is laden with the powdered medicament 168A.

Similarly, the second atrium outlet 178B of the second atrium 172B is in fluid communication with the fourth stack inlet 188 to define the second entrainment airflow path 198 associated with an open blister pocket 164B of the second blister strip 160B. As the airstream flows through the open blister pocket 164B, it picks up the powdered medicament 168B disposed within the open blister pocket 164B. Thus, by passing through the open blister pocket 164B, the powdered medicament 168B is drawn in and transported by the airstream into the stack 180. After entrainment, the airstream is laden with the powdered medicament 168B.

The first atrium outlet 176A of the first atrium 172A is in fluid communication with the first stack inlet 182 to define the first diversion airflow path 192 of the manifold 114. Similarly, the first atrium outlet 176B of the second atrium 172B is in fluid communication with the third stack inlet 186 to define the second diversion airflow path 194 of the manifold 114. Each of the diversion airflow paths 192, 194 provides a lower resistance pathway for air to flow from outside the inhaler device 100 to the patient's mouth compared to the first and second entrainment airflow paths 196, 198. As a result, the overall airflow resistance of the inhaler device 100 is lowered so that higher overall flow rates can be achieved for the same inhalation pressure. In addition, the diversion airflow paths 192, 194 provide de-agglomeration of the powdered medicaments 168A, 168B before they exit the manifold 114. Each of the first and second diversion airflow paths 192, 194 is configured to disrupt each of the first and second entrainment airflow paths 196, 198 and break up medicament carried thereby. More particularly, the first diversion airflow path 192 is directed into the path of the first entrainment airflow path 196, which is at a different angle from the first diversion airflow path 192. A region of higher shear is created at the point of intersection between the first diversion airflow path 192 and the first entrainment airflow path 196, improving de-agglomeration of the powdered medicament 168A before it exits the manifold 114. Similarly, the second diversion airflow path 194 is directed into the path of the second entrainment airflow path 198, which is at a different angle from the second diversion airflow path 194. A region of higher shear is created at the point of intersection between the second diversion airflow path 194 and the second entrainment airflow path 198, improving de-agglomeration of the powdered medicament 168B before it exits the manifold 114. The first diversion airflow path 192, the second diversion airflow path 194, the first entrainment airflow path 196, and the second entrainment airflow path 198 combine or mix together within the stack 180 before exiting the manifold 114.

As represented in the flowchart of FIG. 13B, the inhalation airstream drawn from outside the inhaler device 100 is divided between the two inlets of the manifold 114, namely the first atrium inlet 174A and the second atrium inlet 174B depicted in FIGS. 12 and 13A. The inhalation airstream drawn from outside the inhaler device thus simultaneously enters each of the first atrium 172A and the second atrium 172B. A first portion of the inhalation airstream entering the first atrium 172A flows into the open blister pocket 164A and a second portion of the inhalation airstream entering the first atrium 172A directly flows into the stack 180. The first portion of the inhalation airstream within the open blister pocket 164A picks up or entrains powdered medicament 168A disposed within the open blister pocket 164A, and then continues into the stack 180. Within the stack 180, the second portion of the inhalation airstream from the first atrium 172A breaks up or de-agglomerates the powdered medicament 168A entrained within the first portion of the inhalation airstream. Similarly, at the same time, a first portion of the inhalation airstream entering the second atrium 172B flows into the open blister pocket 164B and a second portion of the inhalation airstream entering the second atrium 172B flows into the stack 180. The first portion of the inhalation airstream within the open blister pocket 164B picks up or entrains powdered medicament 168B disposed within the open blister pocket 164B, and then continues into the stack 180. Within the stack 180, the second portion of the inhalation airstream from the second atrium 172B breaks up or de-agglomerates the powdered medicament 168B entrained within the first portion of the inhalation airstream. Within the stack 180, all portions of the airstream mix together before exiting the manifold 114 towards a patient's mouth, with the combined airstream including both medicament 168A from the first blister strip 160A and medicament 168B from the second blister strip 160B. Airflow through the first atrium 172A and the open blister pocket 164A is concurrent to airflow through the second atrium 172B and the open blister pocket 164B.

Due to the fact that the first and second atriums 172A, 172B are separate and distinct compartments with the divider wall 173 extending therebetween, the inhalation airstream entering into the manifold 114 is directed towards the outlets of each atrium. Directing the separated inhalation airstream into the open blister pockets 164A, 164B in this way reduces turbulent energy in the airflow at this stage, and therefore reduces overall airflow resistance of the inhaler device 100. Overall airflow resistance allows patients to achieve higher flow rates for the same inhalation pressure, which may improve efficacy of drug delivery.

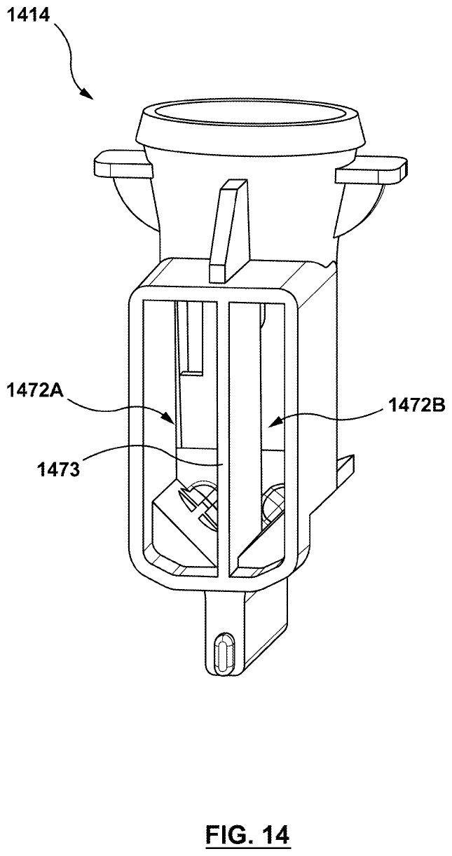

In the embodiment of FIG. 12, the first atrium 172A and the second atrium 172B are the same or substantially similar in volume or size such that an airflow resistance through each atrium is substantially similar. However, in another embodiment depicted in FIG. 14, a manifold 1414 includes a first atrium 1472A and a second atrium 1472B which are different in volume or size such that a first airflow resistance through the first atrium 1472A is lower than a second airflow resistance through the second atrium 1472B. Different atrium sizes are beneficial when it is desired to adjust the relative airflow properties of the first and second entrainment airflow paths. For example, if the powdered medicament 168A in open blister pocket 164A requires a higher flow rate than the powdered medicament 168B in open blister pocket 164B, the position and/or geometry of a divider wall 1473 may be adjusted to optimize the relative resistance of the first and second entrainment airflow paths in order to maximize the overall efficacy of medicament delivery. Keeping the airstreams entering into the manifold 1414 separate and independent from each other allows the relative airflow properties for each pocket 164A, 164B to be easily adjusted, because the size or volume of the atriums may be optimized for different variants of medicament formulations or blister pocket geometry.

Although the manifold 114 is shown with two diversion airflow paths 192, 194 therethrough, it will be apparent to one of ordinary skill in the art that the manifold 114 may include additional diversion airflow paths between the respective atrium and the stack 180. For example, rather than providing a single diversion airflow path between each of the first and second atriums 172A, 172B and the stack 180, each atrium 172A, 172B may include one or more additional outlets to create one or more additional diversion airflow paths directly to the stack 180 without departing from the scope of this invention.

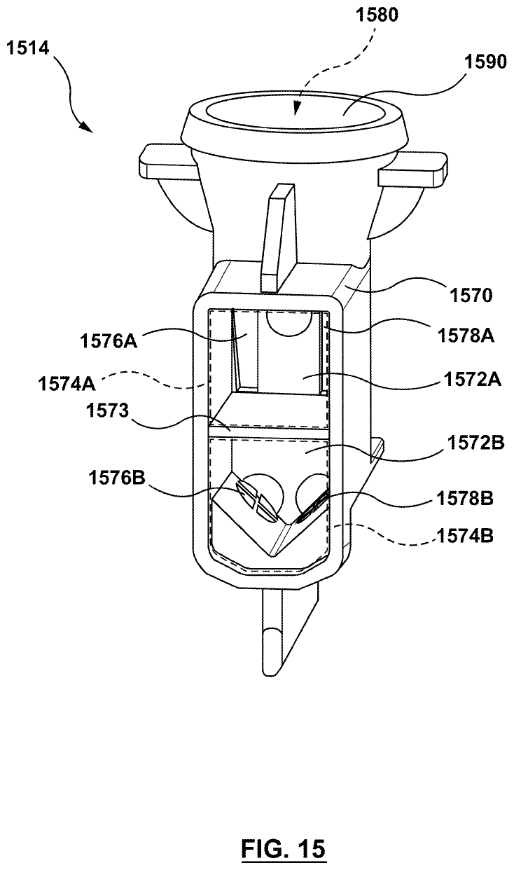

FIGS. 15-17 illustrate another embodiment of a manifold 1514 for use in an inhaler device. In this embodiment, the manifold 1514 includes two atriums, namely an upper atrium and a lower atrium, with the upper atrium having two outlets which form diversion airflow paths and the lower atrium having two outlets which form entrainment airways paths. The relative terms “upper” and “lower” are utilized herein for sake of illustration only, and relate to how an inhaler device is customarily positioned by a user during use with an upper portion of the inhaler device including the mouthpiece.

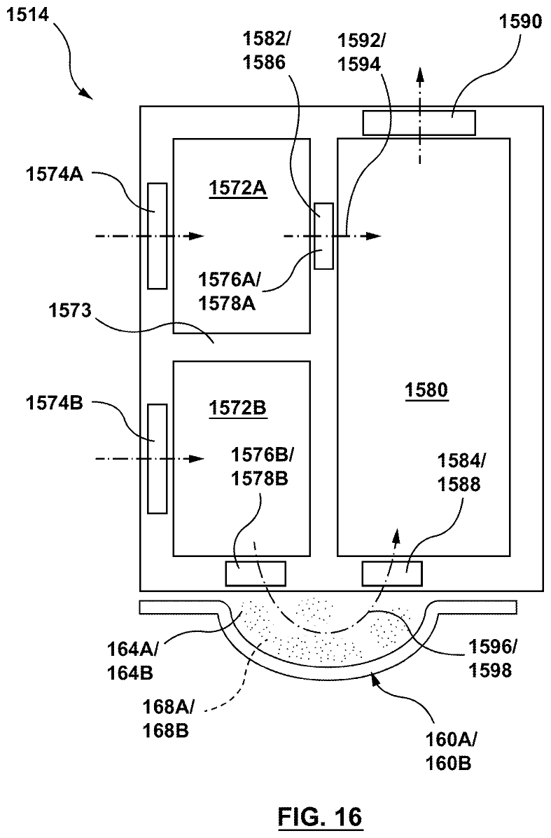

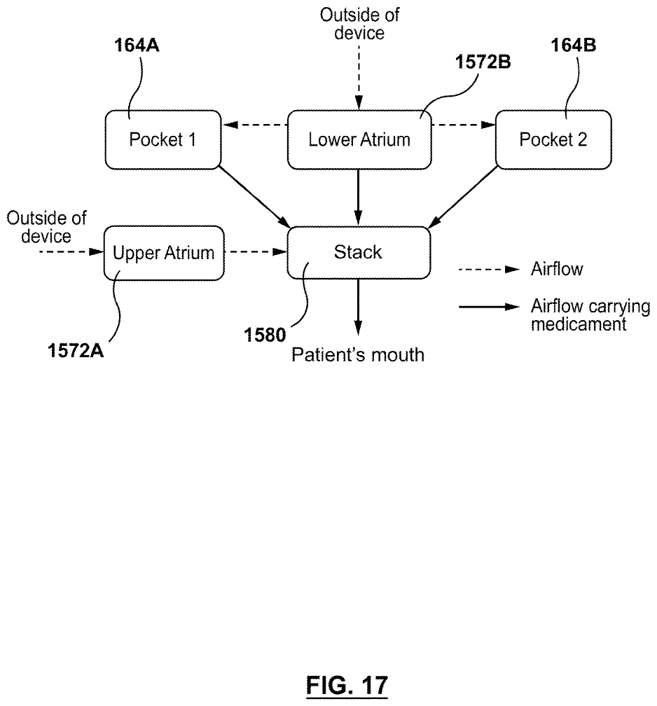

With reference to FIGS. 15-17, the manifold 1514 will be described in more detail. FIG. 15 is a perspective view of the manifold 1514 removed from an inhaler device for sake of illustration only. FIG. 16 is a schematic view illustrating an airflow path through the manifold 1514, and FIG. 17 is a schematic flowchart illustrating the airflow path through the manifold 1514.

The manifold 1514 is configured for simultaneous delivery of the powdered medicaments 168A, 168B from respective open blister pockets 164A, 164B of each of the first blister strip 160A and the second blister strip 160B, respectively. The manifold 1514 includes a body 1570 that defines a first or upper space or atrium 1572A, a second or lower space or atrium 1572B, and a stack 1580. As will be explained in more detail herein, the separated distinct compartments or spaces of the first atrium 1572A, the second atrium 1572B, and the stack 1580 split up, divide, or otherwise separate an inhalation airflow that is drawn into the manifold 1514 into multiple airflow paths through the body 1570 of the manifold 1514. More specifically, when an inhalation force is applied through the opening of a mouthpiece of an inhaler device, an inhalation airflow is drawn into the manifold 1514 via inlet vents of the inhaler device, the inhalation airflow splits or divides into four airflow paths as it travels through the body 1570 of the manifold, namely, a first diversion airflow path 1592, a second diversion airflow path 1594, a first entrainment airflow path 1596, and a second entrainment airflow path 1598. Stated another way, each of the first diversion airflow path 1592, the second diversion airflow path 1594, the first entrainment airflow path 1596, and the second entrainment airflow path 1598 are respective airflow portions of the inhalation airflow which is drawn into the manifold 1514.

In this embodiment, the first atrium 1572A and the second atrium 1572B are disposed vertically adjacent to each other, with one above the other, on a single side of the body 1570 of the manifold 1514. The first atrium 1572A and the second atrium 1572B are separated from each other by a divider wall 1573 such that the first atrium 1572A is not in fluid communication with the second atrium 1572B. Each of the first atrium 1572A and the second atrium 1572B includes a single atrium inlet 1574A, 1574B, respectively. The atrium inlets 1574A, 1574B may also be considered the inlets of the manifold 1514. As such, the manifold 1514 includes only two inlets, the atrium inlet 1574A leading or entering into the first atrium 1572A and the atrium inlet 1574B leading or entering into the second atrium 1572B.

The first atrium 1572A includes at least one atrium outlet, while the second atrium 1572B includes two atrium outlets. In the embodiment of FIGS. 15-17, each of the first atrium 1572A and the second atrium 1572B includes two atrium outlets, namely, a first atrium outlet 1576A, 1576B, respectively, and a second atrium outlet 1578A, 1578B, respectively. As will be explained in more detail herein, the first and second atrium outlets 1576A, 1578A of the first atrium 1572A directs flow into the stack 1580 and the first and second atrium outlets 1576B, 1578B of the second atrium 1572B directs flow into the open pockets 164A, 164B, respectively, of the first and second blister strips 160A, 160B, respectively. In an embodiment, the profile or shape of the first and second atrium outlets 1576A, 1578A of the first atrium 1572A is substantially rectangular or oblong. However, the profile or shape of the first and second atrium outlets 1576A, 1578A of the first atrium 1572A is not limited to the shape depicted herein and may alternatively be circular, triangular, or any other shape deemed suitable for the purposes described herein. Similarly, the profile or shape of the first and second atrium outlets 1576B, 1578B of the second atrium 1572B is substantially circular and includes a grill or cross-piece spanning thereover to encourage increased turbulence in the airflow. However, the profile or shape of the first and second atrium outlets 1576B, 1578B of the second atrium 1572B is not limited to the shape depicted herein and may alternatively be rectangular, oblong, oval, triangular, or any other shape deemed suitable for the purposes described herein and may or may not include a grill spanning thereover.

As best shown on FIG. 16, the stack 1580 is in fluid communication with each of the first atrium 1572A, the open pocket 164A of the first blister strip 160A, and the second open pocket 164B of the second blister strip 160B. In embodiments hereof, a stack may include at least three stack inlets. In the embodiment of FIGS. 15-17, the stack 1580 has four inlets, namely, a first stack inlet 1582, a second stack inlet 1584, a third stack inlet 1586, and a fourth stack inlet 1588. The first stack inlet 1582 is aligned with the first atrium outlet 1576A of the first atrium 1572A, such that the stack 1580 and the first atrium 1572A are in fluid communication with each other. The second stack inlet 1584 is in fluid communication with the open pocket 164A of the first blister strip 160A, such that the second stack inlet 1584 is further in fluid communication with the first atrium outlet 1576B of the second atrium 1572B via the open pocket 164A. The third stack inlet 1586 is aligned with the second atrium outlet 1578A of the first atrium 1572A, such that the stack 1580 and the first atrium 1572A are in further fluid communication with each other. The fourth stack inlet 1588 is in fluid communication with the open pocket 164B of the second blister strip 160B, such that the fourth stack inlet 1588 is further in fluid communication with the second atrium outlet 1578B of the second atrium 1572B via the open pocket 164B.

The stack 1580 is a single stack with a single stack outlet 1590. Thus, the stack 1580 has only one stack outlet 1590. The stack outlet 1590 may also be considered the outlet of the manifold 1514. As such, the manifold 1514 includes a single outlet. A shape or profile of the stack outlet 1590 is oval. However, the profile or shape of the stack outlet 1590 is not limited to the shape depicted herein and may alternatively be circular, rectangular, oblong, triangular, or any other shape deemed suitable for the purposes described herein. When the manifold 1514 is assembled into an inhaler device, the stack outlet 1590 is aligned and in fluid communication with the opening of a mouthpiece of an inhaler device.

The first atrium outlet 1576B of the second atrium 1572B is in fluid communication with the second stack inlet 1584 to define a first entrainment airflow path 1596 associated with an open blister pocket 164A of the first blister strip 160A. As the airstream flows through the open blister pocket 164A, it picks up the powdered medicament 168A disposed within the open blister pocket 164A. Thus, by passing through the open blister pocket 164A, the powdered medicament 168A is drawn in and transported by the airstream into the stack 1580. After entrainment, the airstream is laden with the powdered medicament 168A.

Similarly, the second atrium outlet 1578B of the second atrium 1572B is in fluid communication with the fourth stack inlet 1588 to define a second entrainment airflow path 1598 associated with an open blister pocket 164B of the second blister strip 160B. As the airstream flows through the open blister pocket 164B, it picks up the powdered medicament 168B disposed within the open blister pocket 164B. Thus, by passing through the open blister pocket 164B, the powdered medicament 168B is drawn in and transported by the airstream into the stack 1580. After entrainment, the airstream is laden with the powdered medicament 168B.

The first atrium outlet 1576A of the first atrium 1572A is in fluid communication with the first stack inlet 1582 to define a first diversion airflow path 1592 of the manifold 1514. Similarly, the second atrium outlet 1578A of the first atrium 1572A is in fluid communication with the third stack inlet 1586 to define a second diversion airflow path 1594 of the manifold 1514. Each of the diversion airflow paths 1592, 1594 provides a lower resistance pathway for air to flow from outside the inhaler device 100 to the patient's mouth compared to the first and second entrainment airflow paths 1596, 1598. As a result, the overall airflow resistance of the inhaler device 100 is lowered so that higher overall flow rates can be achieved for the same inhalation pressure. In addition, the diversion airflow paths 1592, 1594 provide de-agglomeration of the powdered medicaments 168A, 168B before they exit the manifold 1514. Each of the first and second diversion airflow paths 1592, 1594 is positioned and configured to disrupt each of the first and second entrainment airflow paths 1596, 1598, respectively, and break up powdered medicament carried thereby. More particularly, the first diversion airflow path 1592 is directed into the path of the first entrainment airflow path 1596, which is at a different angle from the first diversion airflow path 1592. A region of higher shear is created at the point of intersection between the first diversion airflow path 1592 and the first entrainment airflow path 1596, improving de-agglomeration of the powdered medicament 168A before it exits the manifold 1514. Similarly, the second diversion airflow path 1594 is directed into the path of the second entrainment airflow path 1598, which is at a different angle from the second diversion airflow path 1594. A region of higher shear is created at the point of intersection between the second diversion airflow path 1594 and the second entrainment airflow path 1598, improving de-agglomeration of the powdered medicament 168B before it exits the manifold 1514. The first diversion airflow path 1592, the second diversion airflow path 1594, the first entrainment airflow path 1596, and the second entrainment airflow path 1598 combine or mix together within the stack 1580 before exiting the manifold 1514.

As represented in the flowchart of FIG. 17, the inhalation airstream drawn from outside an inhaler device is divided between the two inlets of the manifold 1514, namely the first atrium inlet 1574A and the second atrium inlet 1574B. The inhalation airstream drawn from outside the inhaler device thus simultaneously enters each of the first atrium 1572A and the second atrium 1572B. A first portion of the inhalation airstream entering the first atrium 1572A flows into the stack 1580 and a second portion of the inhalation airstream entering the first atrium 1572A flows into the stack 1580. Similarly, at the same time, a first portion of the inhalation airstream entering the second atrium 1572B flows into the open blister pocket 164A and a second portion of the inhalation airstream entering the second atrium 1572B flows into the open blister pocket 164B. The first portion of the inhalation airstream within the open blister pocket 164A picks up or entrains medicament 168A disposed within the open blister pocket 164A, and then continues into the stack 1580. The second portion of the inhalation airstream within the open blister pocket 164B picks up or entrains medicament 168B disposed within the open blister pocket 164B, and then continues into the stack 1580. Within the stack 1580, the first portion of the inhalation airstream from the first atrium 1572A breaks up or de-agglomerates the powdered medicament 168A entrained within the first portion of the inhalation airstream and the second portion of the inhalation airstream from the first atrium 1572A breaks up or de-agglomerates the powdered medicament 168B entrained within the second portion of the inhalation airstream. Within the stack 1580, all portions of the airstream mix together before exiting the manifold 1514 towards a patient's mouth, with the combined airstream including both medicament 168A from the first blister strip 160A and medicament 168B from the second blister strip 160B.

Due to the fact that the first and second atriums 1572A, 1572B are separate and distinct compartments with the divider wall 1573 extending therebetween, the inhalation airstream entering into the manifold 1514 is directed towards the outlets of each atrium. Directing the separated inhalation airstream into the open blister pockets 164A, 164B in this way allows for more independent control over the turbulent energy in the airflow at this stage, and therefore allows for more control over the overall airflow resistance of the inhaler device 100. Overall airflow resistance allows patients to achieve higher flow rates same inhalation for the pressure, which may improve efficacy of drug delivery.

Separating the entrainment airflow paths 1596, 1598 from the diversion airflow paths 1592, 1594 makes it easier to control the proportion or amount of airflow that enters the open blister pockets relative to the total inlet airflow, which is an important parameter in optimizing efficacy of drug delivery. In the embodiment of FIG. 15, the first atrium 1572A and the second atrium 1572B are the same or substantially similar in volume or size such that an airflow resistance through each atrium is substantially similar. However, in another embodiment, the first atrium 1572A and the second atrium 1572B are different in volume or size such that a first airflow resistance through the first atrium 1572A is lower than a second airflow resistance through the second atrium 1572B. Different atrium sizes are beneficial when it is desired to adjust the airflow properties of the diversion airflow paths 1592, 1594. For example, if a greater amount of de-agglomeration is required for particular medicaments, the position and/or geometry of the divider wall 1573 may be adjusted to optimize the relative resistance of entrainment airflow paths 1596, 1598 compared to the diversion airflow paths 1592, 1594, in order to maximize the overall efficacy of medicament delivery. Keeping the entrainment and diversion airstreams entering into the manifold 1514 separate and independent from each other allows the airflow properties of the two airstreams to be independently controlled, because the size or volume of the atriums may be optimized for different variants of medicament formulations or blister pocket geometry. For example, it may be desirable to achieve different levels of turbulent energy in a particular airstream in order to improve entrainment and/or de-agglomeration of a medicament.

Although the manifold 1514 is shown with two diversion airflow paths 1592, 1594 therethrough, it will be apparent to one of ordinary skill in the art that the manifold 1514 may include only a single diversion airflow path between the first atrium 1572A and the stack 1580, or may include additional (i.e., more than two) diversion airflow paths between the first atrium 1572A and the stack 1580.

Although the manifold 1514 is shown with two atriums, namely the first atrium 1572A and the second atrium 1572B, in another embodiment hereof (not shown) each of the first and second atriums 1572A, 1572B may include a vertical divider wall to form a total of four separate atriums. As such, the manifold thereof would include a total of four inlets and each manifold inlet would be associated with one of the outlets 1576A, 1576B, 1578A, 1578B. Stated another way, the first and second atriums 1572A, 1572B may be split or subdivided so that each of the four airflow paths of the manifold has a separate or dedicated manifold inlet.

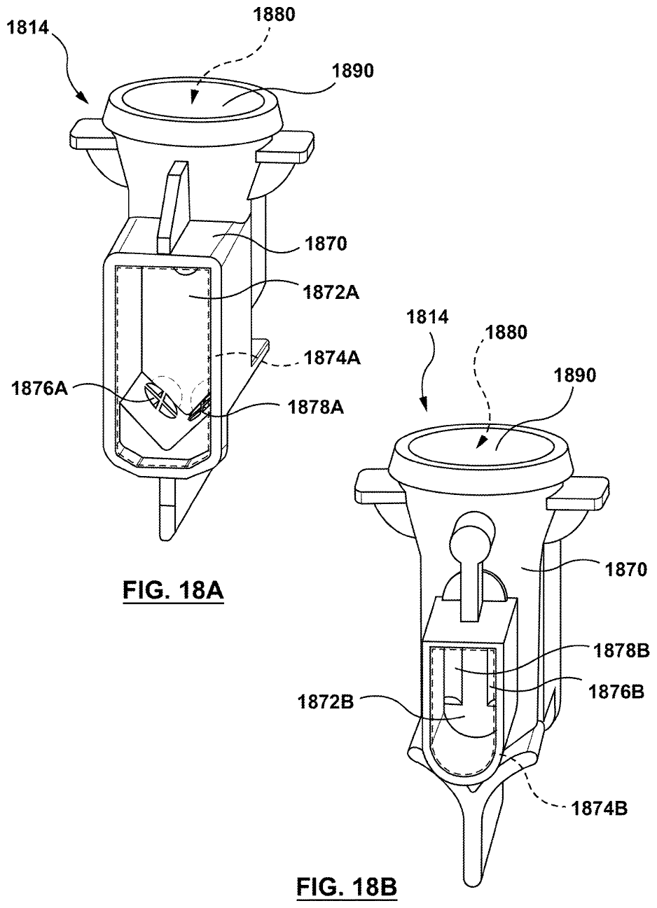

FIGS. 18A-21B illustrate another embodiment of a manifold 1814 for use in an inhaler device. In this embodiment, the manifold 1814 includes two atriums, namely a front atrium and a rear atrium, with the front atrium having two outlets which form entrainment airflow paths and the rear atrium having two outlets which form diversion airflow paths. The relative terms “front” and “rear” are utilized herein for sake of illustration only, and relate to how an inhaler device is customarily positioned by a user during use with a front of the inhaler device including the display window for the counter mechanism. In this embodiment, the total inlet area is increased since both sides of the manifold are utilized, allowing for reduced total airflow resistance of the inhaler device.

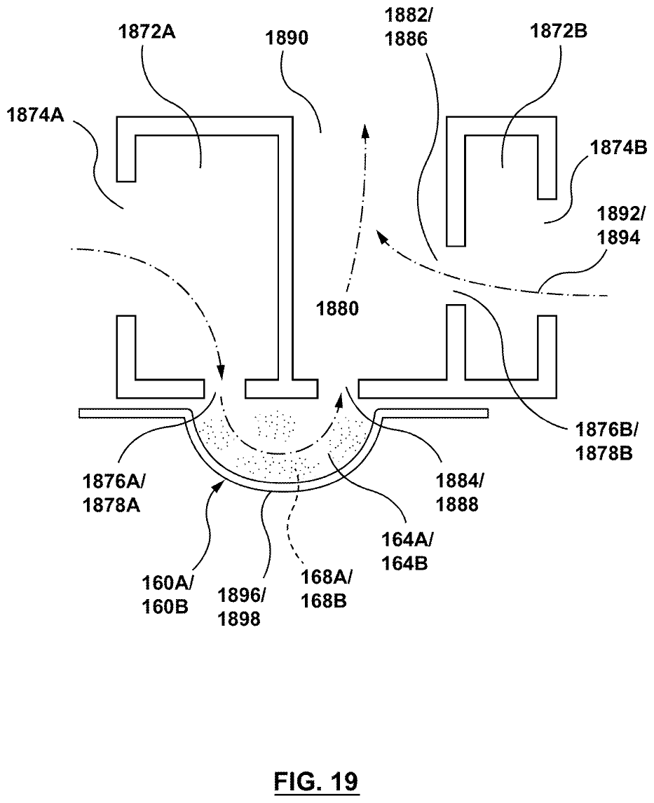

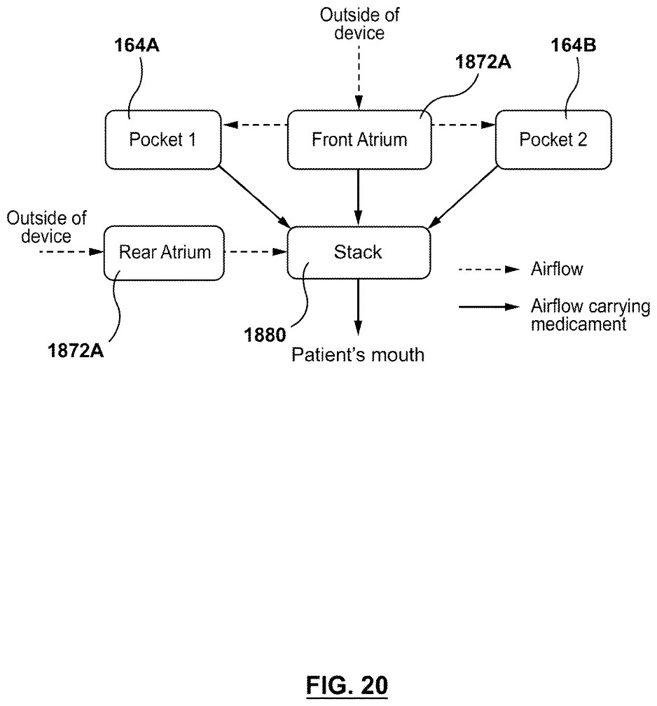

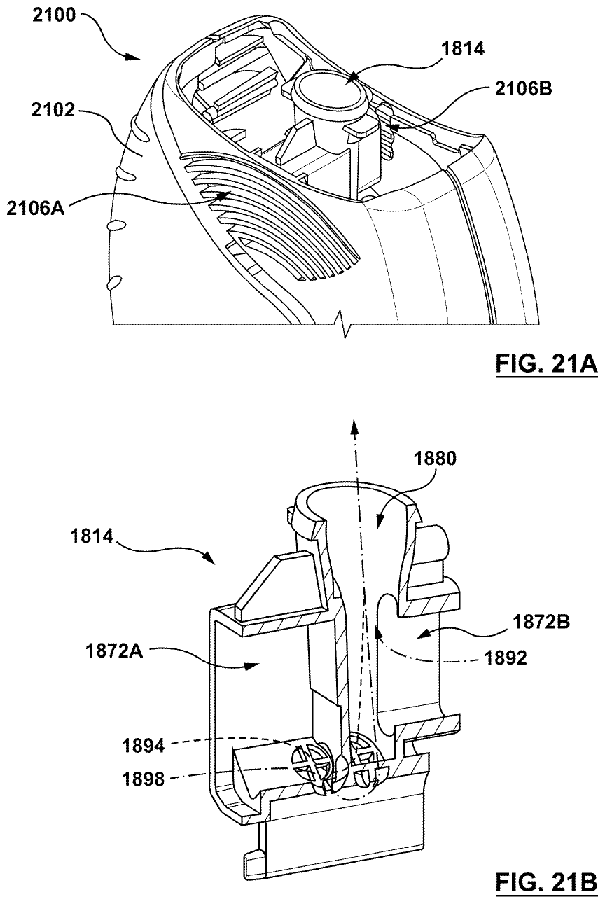

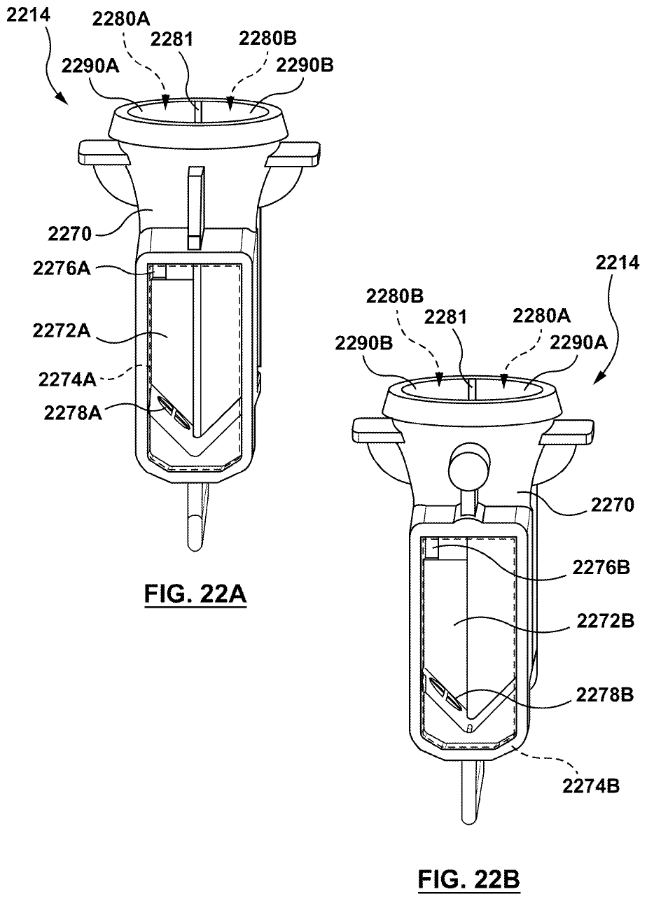

With reference to FIGS. 18A-21B, the manifold 1814 will be described in more detail. FIGS. 18A and 18B are perspective views of opposing front and rear sides of the manifold 1814, with the manifold removed from the inhaler device for sake of illustration only. FIG. 19 is a schematic view illustrating the airflow path through the manifold 1814, and FIG. 20 is a schematic flowchart illustrating the airflow path through the manifold 1814. FIG. 21A is a perspective view illustrating placement of the manifold 1814 within a housing 2102 of an inhaler device 2100, and FIG. 22B is a perspective sectional view of the manifold 1814.

The manifold 1814 is configured for simultaneous delivery of the powdered medicaments 168A, 168B from respective open blister pockets 164A, 164B, of each of the first blister strip 160A and the second blister strip 160B, respectively. The manifold 1814 includes a body 1870 that defines a first or front space or atrium 1872A, a second or rear space or atrium 1872B, and a stack 1880. As will be explained in more detail herein, the separated distinct compartments or spaces of the first atrium 1872A, the second atrium 1872B, and the stack 1880 split up, divide, or otherwise separate an inhalation airflow that is drawn into the manifold 1814 into multiple airflow paths through the body 1870 of the manifold 1814. More specifically, when an inhalation force is applied through the opening of a mouthpiece of an inhaler device, an inhalation airflow is drawn into the manifold 1814 via inlet vents of the inhaler device, the inhalation airflow splits or divides into four airflow paths as it travels through the body 1870 of the manifold, namely, a first diversion airflow path 1892, a second diversion airflow path 1894, a first entrainment airflow path 1896, and a second entrainment airflow path 1898. Stated another way, each of the first diversion airflow path 1892, the second diversion airflow path 1894, the first entrainment airflow path 1896, and the second entrainment airflow path 1898 are respective airflow portions of the inhalation airflow which is drawn into the manifold 1814.

In this embodiment, the first atrium 1872A and the second atrium 1872B are disposed on opposing sides of the body 1870 of the manifold 1814, with the stack 1880 disposed between the first and second atriums 1872A, 1872B. The first atrium 1872A and the second atrium 1872B are separated from each other such that the first atrium 1872A is not in fluid communication with the second atrium 1872B. With reference to FIG. 21A, when the manifold 1814 is disposed within the housing 2102 of the inhaler device 2100, the atrium 1872A is disposed adjacent to vent openings 2106A and the atrium 1872B is disposed adjacent to vent openings 2106B, wherein each set of vent openings 2106A, 2106B is formed within opposite sides of the housing 2102. Thus, in this embodiment, the housing 2102 of the inhaler device 2100 includes vent openings 2106A, 2106B on opposing sides thereof rather than a single side as in previous embodiments.

Each of the first atrium 1872A and the second atrium 1872B includes a single atrium inlet 1874A, 1874B, respectively. The atrium inlets 1874A, 1874B may also be considered the inlets of the manifold 1814. As such, the manifold 1814 includes two inlets, the atrium inlet 1874A leading or entering into the first atrium 1872A and the atrium inlet 1874B leading or entering into the second atrium 1872B. Compared to manifolds 114 and 1514, which has the first and second atriums on the same side of the manifold, the size of the atrium inlets 1874A, 1874B is greater or increased since the atriums 1872A, 1872B are disposed on opposing sides of the body 1870 of the manifold 1814. Opposing sides of the manifold 1814 have more available space to permit larger atrium inlets. A greater total inlet area may reduce overall device resistance.

In the embodiment of FIGS. 18A-21B, each of the first atrium 1872A and the second atrium 1872B includes two atrium outlets, namely, a first atrium outlet 1876A, 1876B, respectively, and a second atrium outlet 1878A, 1878B, respectively. As will be explained in more detail herein, the first and second atrium outlets 1876A, 1878A of the first atrium 1872A guide or direct flow into the open pockets 164A, 164B, respectively, of the first and second blister strips 160A, 160B, respectively, and the first and second atrium outlets 1876B, 1878B of the second atrium 1872B guide or direct flow directly into the stack 1880. In an embodiment, the profile or shape of the first and second atrium outlets 1876B, 1878B of the second atrium 1872B is substantially rectangular or oblong. However, the profile or shape of first and second atrium outlets 1876B, 1878B of the second atrium 1872B is not limited to the shape depicted herein and may alternatively be circular, triangular, or any other shape deemed suitable for the purposes described herein. Similarly, the profile or shape of the first and second atrium outlets 1876A, 1878A of the first atrium 1872A is substantially circular and includes a grill or cross-piece spanning thereover to encourage increased turbulence in the airflow. However, the profile or shape of the first and second atrium outlets 1876A, 1878A of the first atrium 1872A is not limited to the shape depicted herein and may alternatively be rectangular, oblong, oval, triangular, or any other shape deemed suitable for the purposes described herein and may or may not include a grill spanning thereover.

As best shown on FIG. 19, the stack 1880 is in fluid communication with each of the second atrium 1872B, the open pocket 164A of the first blister strip 160A, and the second open pocket 164B of the second blister strip 160B. In the embodiment of FIGS. 18A-21B, the stack 1880 has four inlets, namely, a first stack inlet 1882, a second stack inlet 1884, a third stack inlet 1886, and a fourth stack inlet 1888. The first stack inlet 1882 is aligned with the first atrium outlet 1876B of the second atrium 1872B, such that the stack 1880 and the second atrium 1872B are in fluid communication with each other. The second stack inlet 1884 is in fluid communication with the open pocket 164A of the first blister strip 160A, such that the second stack inlet 1884 is further in fluid communication with the first atrium outlet 1876A of the first atrium 1872A via the open pocket 164A. The third stack inlet 1886 is aligned with the second atrium outlet 1878B of the second atrium 1872B, such that the stack 1880 and the second atrium 1872B are in further fluid communication with each other. The fourth stack inlet 1888 is in fluid communication with the open pocket 164B of the second blister strip 160B, such that the fourth stack inlet 1888 is further in fluid communication with the second atrium outlet 1878A of the first atrium 1872A via the open pocket 164B.

The stack 1880 is a single stack with a single stack outlet 1890. Stated another way, the stack 1880 has only one stack outlet 1890. The stack outlet 1890 may also be considered the outlet of the manifold 1814. As such, the manifold 1814 includes only one outlet. A shape or profile of the stack outlet 1890 is oval. However, the profile or shape of the stack outlet 1890 is not limited to the shape depicted herein and may alternatively be circular, rectangular, oblong, triangular, or any other shape deemed suitable for the purposes described herein. When the manifold 1814 is assembled into an inhaler device, the stack outlet 1890 is aligned and in fluid communication with the opening of the mouthpiece of the inhaler device.

The first atrium outlet 1876A of the first atrium 1872A is in fluid communication with the second stack inlet 1884 to define a first entrainment airflow path 1896 associated with an open blister pocket 164A of the first blister strip 160A. As the airstream flows through the open blister pocket 164A, it picks up the powdered medicament 168A disposed within the open blister pocket 164A. Thus, by passing through the open blister pocket 164A, the powdered medicament 168A is drawn in and transported by the airstream into the stack 1880. After entrainment, the airstream is laden with the powdered medicament 168A.

Similarly, the second atrium outlet 1878A of the first atrium 1872A is in fluid communication with the fourth stack inlet 1888 to define a second entrainment airflow path 1898 associated with an open blister pocket 164B of the second blister strip 160B. As the airstream flows through the open blister pocket 164B, it picks up the powdered medicament 168B disposed within the open blister pocket 164B. Thus, by passing through the open blister pocket 164B, the powdered medicament 168B is drawn in and transported by the airstream into the stack 1880. After entrainment, the airstream is laden with the powdered medicament 168B.

The first atrium outlet 1876B of the second atrium 1872B is in fluid communication with the first stack inlet 1882 to define a first diversion airflow path 1892 of the manifold 1814. Similarly, the second atrium outlet 1878B of the second atrium 1872B is in fluid communication with the third stack inlet 1886 to define a second diversion airflow path 1894 of the manifold 1814. Each of the diversion airflow paths 1892, 1894 provides a lower resistance pathway for air to flow from outside the inhaler device 2100 to the patient's mouth compared to the first and second entrainment airflow paths 1896, 1898. As a result, the overall airflow resistance of the inhaler device 2100 is lowered so that higher overall flow rates can be achieved for the same inhalation pressure. In addition, the diversion airflow paths 1892, 1894 provide de-agglomeration of the powdered medicaments 168A, 168B before they exit the manifold 1814. Each of the first and second diversion airflow paths 1892, 1894 is positioned and configured to disrupt each of the first and second entrainment airflow paths 1896, 1898 and break up the powdered medicament carried thereby. More particularly, the first diversion airflow path 1892 is directed into the path of the first entrainment airflow path 1896, which is at a different angle from the first diversion airflow path 1892. A region of higher shear is created at the point of intersection between the first diversion airflow path 1892 and the first entrainment airflow path 1896, improving de-agglomeration of the powdered medicament 168A before it exits the manifold 1814. Similarly, the second diversion airflow path 1894 is directed into the path of the second entrainment airflow path 1898, which is at a different angle from the second diversion airflow path 1894. A region of higher shear is created at the point of intersection between the second diversion airflow path 1894 and the second entrainment airflow path 1898, improving de-agglomeration of the powdered medicament 168B before it exits the manifold 1814. The first diversion airflow path 1892, the second diversion airflow path 1894, the first entrainment airflow path 1896, and the second entrainment airflow path 1898 combine or mix together within the stack 1880 before exiting the manifold 1814.

As represented in the flowchart of FIG. 20, the inhalation airstream drawn from outside an inhaler device is divided between the two inlets of the manifold 1814, namely the first atrium inlet 1874A and the second atrium inlet 1874B. The inhalation airstream drawn from outside the inhaler device thus simultaneously enters each of the first atrium 1872A and the second atrium 1872B. A first portion of the inhalation airstream entering the second atrium 1872B flows into the stack 1880 via inlet 1882 and a second portion of the inhalation airstream entering the second atrium 1872B flows into the stack 1880 via inlet 1886. Similarly, at the same time, a first portion of the inhalation airstream entering the first atrium 1872A flows into the open blister pocket 164A and a second portion of the inhalation airstream entering the first atrium 1872A flows into the open blister pocket 164B. The first portion of the inhalation airstream within the open blister pocket 164A picks up or entrains medicament 168A disposed within the open blister pocket 164A, and then continues into the stack 1880. The second portion of the inhalation airstream within the open blister pocket 164B picks up or entrains medicament 168B disposed within the open blister pocket 164B, and then continues into the stack 1880. Within the stack 1880, the first portion of the inhalation airstream from the second atrium 1872B breaks up or de-agglomerates the powdered medicament 168A entrained within the first portion of the inhalation airstream and the second portion of the inhalation airstream from the second atrium 1872B breaks up or de-agglomerates the powdered medicament 168B entrained within the second portion of the inhalation airstream. Within the stack 1880, all portions of the airstream mix together before exiting the manifold 1814 towards a patient's mouth, with the combined airstream including both medicament 168A from the first blister strip 160A and medicament 168B from the second blister strip 160B.

Due to the fact that the first and second atriums 1872A, 1872B are separate and distinct compartments, the inhalation airstream entering into the manifold 1814 is directed towards the outlets of each atrium. Directing the separated inhalation airstream into the open blister pockets 164A, 164B in this way allows for more independent control over the turbulent energy in the airflow at this stage, and therefore allows for more control over the overall airflow resistance of the inhaler device 100. Changing airflow resistance allows patients to achieve different flow rates for the same inhalation pressure, which may improve efficacy of drug delivery.

Separating the entrainment airflow paths 1896, 1898 from the diversion airflow paths 1892, 1894 makes it easier to control the proportion or amount of airflow that enters the open blister pockets relative to the total inlet airflow, which is an important parameter in optimizing efficacy of drug delivery. In the embodiment of FIGS. 18A-21B, the volume and shape of the first atrium 1872A and the second atrium 1872B are different and are configured to control the proportion of airflow going through each atrium so that the first and second atriums 1872A, 1872B have different airflow resistances. Different atrium sizes or shapes are beneficial when it is desired to adjust the airflow properties of the diversion airflow paths 1892, 1894. For example, if a greater amount of de-agglomeration is required for particular medicaments, the size or volume of the second atrium 1872B may be adjusted to optimize the relative resistance of entrainment airflow paths 1896, 1898 compared to the diversion airflow paths 1892, 1894, in order to maximize the overall efficacy of medicament delivery. Keeping the entrainment and diversion airstreams entering into the manifold 1814 separate and independent from each other allows the airflow properties of the two airstreams to be independently controlled, because the size or volume of the atriums may be optimized for different variants of medicament formulations or blister pocket geometry. For example, it may be desirable to achieve different levels of turbulent energy in a particular airstream in order to improve entrainment and/or de-agglomeration of medicament.

Although the manifold 1814 is shown with two diversion airflow paths 1892, 1894 therethrough, it will be apparent to one of ordinary skill in the art that the manifold 1814 may include only a single diversion airflow path between the second atrium 1872B and the stack 1880, or may include additional (i.e., more than two) diversion airflow paths between the second atrium 1872B and the stack 1880. The number of stack inlets may accordingly vary depending on number of diversion airflow paths between the second atrium 1872B and the stack 1880.

FIGS. 22A-25 illustrate another embodiment of a manifold 2214 for use in an inhaler device. In this embodiment, the airflow paths through respective open blister pockets 164A, 164B, of each of the first blister strip 160A and the second blister strip 160B, respectively, are separated completely from each other until exiting the manifold 2214. For each open blister pocket 164A, 164B, there is a dedicated atrium and a dedicated stack such that there is no mixing of the powdered medicaments from open blister pocket 164A, 164B, of each of the first blister strip 160A and the second blister strip 160B, respectively, within the manifold 2214.

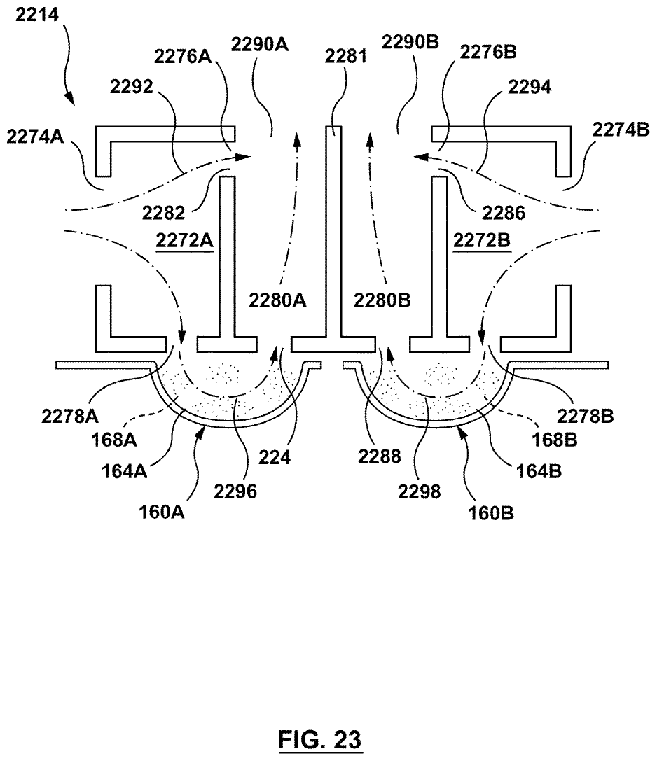

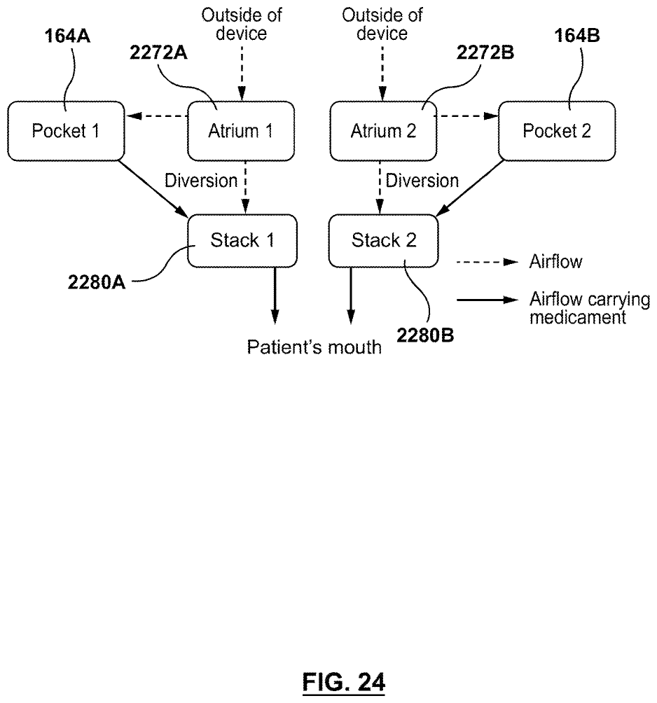

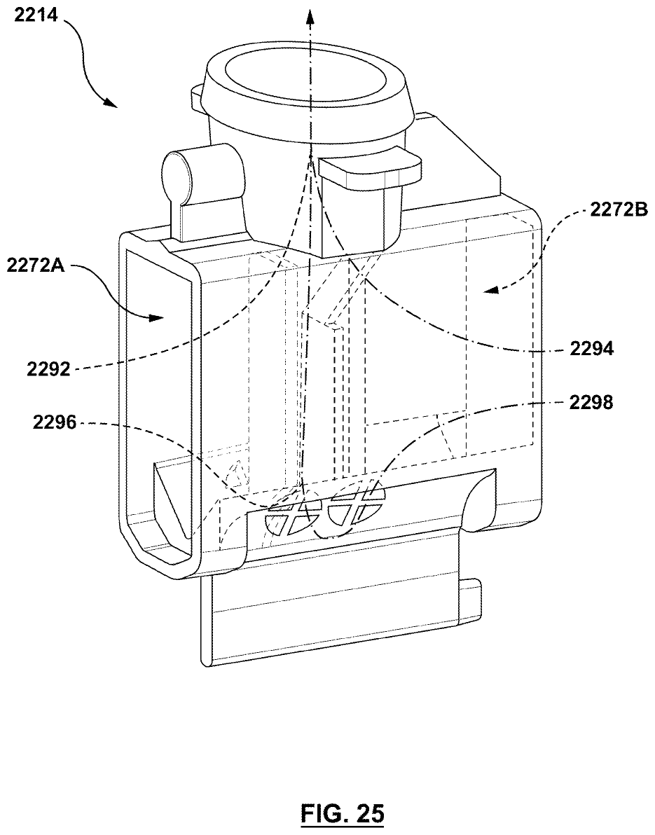

With reference to FIGS. 22A-25, the manifold 2214 will be described in more detail. FIGS. 22A and 22B are front and rear perspective views of the manifold 2214 removed from an inhaler device for sake of illustration only. The relative terms “front” and “rear” are utilized herein for sake of illustration only, and relate to how an inhaler device is customarily positioned by a user during use with a front of the inhaler device including the display window for the counter mechanism. FIG. 23 is a schematic view illustrating the airflow path through the manifold 2214, and FIG. 24 is a schematic flowchart illustrating the airflow path through the manifold 2214. FIG. 25 is a perspective sectional view of the manifold 2214.

The manifold 2214 is configured for simultaneous delivery of powdered medicaments 168A, 168B from respective open blister pockets 164A, 164B, of each of the first blister strip 160A and the second blister strip 160B, respectively. The manifold 2214 includes a body 2270 that defines a first or front space or atrium 2272A, a second or rear space or atrium 2272B, a first stack 2280A, and a second stack 2280B. As will be explained in more detail herein, the separated distinct compartments or spaces of the first atrium 2272A, the second atrium 2272B, the first stack 2280A, and the second stack 2280B split up, divide, or otherwise separate an inhalation airflow that is drawn into the manifold 2214 into multiple airflow paths through the body 2270 of the manifold 2214. More specifically, when an inhalation force is applied through the opening 2212 of the mouthpiece 2210, an inhalation airflow is drawn into the manifold 2214 via respective inlet vents of the inhaler device, the inhalation airflow splits into four airflow paths as it travels through the body 2270 of the manifold, namely, a first diversion airflow path 2292, a second diversion airflow path 2294, a first entrainment airflow path 2296, and a second entrainment airflow path 2298. Stated another way, each of the first diversion airflow path 2292, the second diversion airflow path 2294, the first entrainment airflow path 2296, and the second entrainment airflow path 2298 are respective airflow portions of the inhalation airflow which is drawn into the manifold 2214.

In this embodiment, the first atrium 2272A and the second atrium 2272B are disposed on opposing sides of the body 2270 of the manifold 2214. The first atrium 2272A and the second atrium 2272B are separated from each other such that the first atrium 2272A is not in fluid communication with the second atrium 2272B. Similar to the housing 2102 described above for use with the manifold 1814, when the manifold 2214 is disposed within a housing of an inhaler device, each atrium 2272A, 2272B is disposed adjacent to vent openings formed on opposing sides of the housing.

Each of the first atrium 2272A and the second atrium 2272B includes a single atrium inlet 2274A, 2274B, respectively. The atrium inlets 2274A, 2274B may also be considered the inlets of the manifold 2214. As such, the manifold 2214 includes two inlets, the atrium inlet 2274A leading or entering into the first atrium 2272A and the atrium inlet 2274B leading or entering into the second atrium 2272B. Compared to the manifolds 114 and 1514, which have the first and second atriums on the same side of the manifold, the size of the atrium inlets 2274A, 2274B is greater or increased since the atriums 2272A, 2272B are disposed on opposing sides of the body 2270 of the manifold 2214. Opposing sides of the manifold 2214 have more available space to permit larger atrium inlets. A greater total inlet area may reduce overall device resistance.

Each of the first atrium 2272A and the second atrium 2272B includes two atrium outlets, namely, a first atrium outlet 2276A, 2276B, respectively, and a second atrium outlet 2278A, 2278B, respectively. As will be explained in more detail herein, the first atrium outlet 2276A, 2276B of each of the first and second atriums 2272A, 2272B, respectively, directs air flow into stacks 2280A, 2280B, respectively, and the second atrium outlet 2278A, 2278B of each of the first and second atriums 2272A, 2272B, respectively, directs air flow into the open pocket 164A, 164B of the first and second blister strips 160A, 160B. In an embodiment, the profile or shape of the first atrium outlet 2276A, 2276B is substantially rectangular or oblong. However, the profile or shape of the first atrium outlets 2276A, 2276B is not limited to the shape depicted herein and may alternatively be circular, triangular, or any other shape deemed suitable for the purposes described herein. Similarly, the profile or shape of the second atrium outlets 2278A, 2278B is substantially circular and includes a grill or cross-piece spanning thereover to encourage increased turbulence in the airflow. However, the profile or shape of the second atrium outlets 2278A, 2278B is not limited to the shape depicted herein and may alternatively be rectangular, oblong, oval, triangular, or any other shape deemed suitable for the purposes described herein and may or may not include a grill spanning thereover.

As best shown on FIG. 23, the first stack 2280A is in fluid communication with the first atrium 2272A and the open pocket 164A of the first blister strip 160A. The first stack 2280A has two inlets, namely, a first stack inlet 2282 and a second stack inlet 2284. The first stack inlet 2282 is aligned with the first atrium outlet 2276A of the first atrium 2272A, such that the first stack 2280A and the first atrium 2272A are in fluid communication with each other. The second stack inlet 2284 is in fluid communication with the open pocket 164A of the first blister strip 160A, such that the second stack inlet 2284 is further in fluid communication with the second atrium outlet 2278A of the first atrium 2272A.

Similarly, the second stack 2280B is in fluid communication with the second atrium 2272B and the open pocket 164B of the second blister strip 160B. The second stack 2280B has two inlets, namely, a first stack inlet 2286 and a second stack inlet 2288. The first stack inlet 2286 is aligned with the first atrium outlet 2276B of the second atrium 2272B, such that the second stack 2280B and the second atrium 2272B are in fluid communication with each other. The second stack inlet 2288 is in fluid communication with the open pocket 164B of the second blister strip 160B, such that the second stack inlet 2288 is further in fluid communication with the second atrium outlet 2278B of the second atrium 2272B.

Each stack 2280A, 2280B has a single stack outlet 2290A, 2290B. The stack outlets 2290A, 2290B may also be considered the outlets of the manifold 2214. As such, the manifold 2214 includes two outlets. A shape or profile of each stack outlet 2290A, 2290B is semicircular. However, the profile or shape of each stack outlet is not limited to the shape depicted herein and may alternatively be any other shape deemed suitable for the purposes described herein. When the manifold 2214 is assembled into an inhaler device, each stack outlet 2290A, 2290B is aligned and in fluid communication with the opening of the mouthpiece of the inhaler device.

The second atrium outlet 2278A of the first atrium 2272A is in fluid communication with the second stack inlet 2284 to define a first entrainment airflow path 2296 associated with an open blister pocket 164A of the first blister strip 160A. As the airstream flows through the open blister pocket 164A, it picks up the powdered medicament 168A disposed within the open blister pocket 164A. Thus, by passing through the open blister pocket 164A, the powdered medicament 168A is drawn in and transported by the airstream into the stack 2280A. After entrainment, the airstream is laden with the powdered medicament 168A.

Similarly, the second atrium outlet 2278B of the second atrium 2272B is in fluid communication with the fourth stack inlet 2288 to define a second entrainment airflow path 2298 associated with an open blister pocket 164B of the second blister strip 160B. As the airstream flows through the open blister pocket 164B, it picks up the powdered medicament 168B disposed within the open blister pocket 164B. Thus, by passing through the open blister pocket 164B, the powdered medicament 168B is drawn in and transported by the airstream into the stack 2280B. After entrainment, the airstream is laden with the powdered medicament 168B.

The first atrium outlet 2276A of the first atrium 2272A is in fluid communication with the first stack inlet 2282 to define a first diversion airflow path 2292 of the manifold 2214. Similarly, the first atrium outlet 2276B of the second atrium 2272B is in fluid communication with the third stack inlet 2286 to define a second diversion airflow path 2294 of the manifold 2214. Each of the diversion airflow paths 2292, 2294 provides a lower resistance pathway for air to flow from outside the inhaler device to the patient's mouth compared to the first and second entrainment airflow paths 2296, 2298. As a result, the overall airflow resistance of the inhaler device is lowered so that higher overall flow rates can be achieved for the same inhalation pressure. In addition, the diversion airflow paths 2292, 2294 provide de-agglomeration of the powdered medicaments 168A, 168B before they exit the manifold 2214. Each of the first and second diversion airflow paths 2292, 2294 is configured to disrupt each of the first and second entrainment airflow paths 2296, 2298 and break up powdered medicament carried thereby. More particularly, the first diversion airflow path 2292 is directed into the path of the first entrainment airflow path 2296 within the first stack 2280A, which is at a different angle from the first diversion airflow path 2292. A region of higher shear is created at the point of intersection between the first diversion airflow path 2292 and the first entrainment airflow path 2296, improving de-agglomeration of the powdered medicament 168A before it exits the manifold 2214. Similarly, the second diversion airflow path 2294 is directed into the path of the second entrainment airflow path 2298 within the second stack 2280B, which is at a different angle from the second diversion airflow path 2294. A region of higher shear is created at the point of intersection between the second diversion airflow path 2294 and the second entrainment airflow path 2298, improving de-agglomeration of the powdered medicament 168B before it exits the manifold 2214.

In this embodiment, the first diversion airflow path 2292 and the first entrainment airflow path 2296 combine or mix together within the first stack 2280A before exiting the manifold 2214. Similarly, the second diversion airflow path 2294 and the second entrainment airflow path 2298 combine or mix together within the second stack 2280B before exiting the manifold 2214. The first diversion airflow path 2292 is configured to disrupt the first entrainment airflow path 2296 and break up medicament carried thereby, and the second diversion airflow path 2294 is configured to disrupt the second entrainment airflow path 2298 and break up medicament carried thereby. However, the airstreams within the first and second stacks 2280A, 2280B, and thus the powdered medicaments carried thereby, do not combine or mix together. Rather, in this embodiment, the first stack 2280A and the second stack 2280B are separated from each other by a divider wall 2281 such that the first stack 2280A is not in fluid communication with the second stack 2280B and mixing of the powdered medicament within the first entrainment airflow path 2296 and the powdered medicament within the second entrainment airflow path 2298 is prevented within the manifold 2214 along a height of the divider wall. In the embodiment of FIGS. 22A-25, the height of the divider wall 2281 extends a full or entire height of the stacks 2280A, 2280B within the manifold 2214 such that the separated airstreams within the first and second stacks 2280A, 2280B do not combine or mix together until exiting from the manifold 2214. Accordingly, the separated airstreams within the first and second stacks 2280A, 2280B and the respective medicaments therein may combine or mix together exteriorly of the inhaler device, such as, within a patient's mouth. Separated stacks 2280A, 2280B are thus beneficial when it is desired to avoid mixing of the powdered medicaments 168A, 168B within the inhaler device.

Due to the fact that the first and second stacks 2280A, 2280B are separate and distinct compartments with the divider wall 2281 extending therebetween, the design of the manifold 2214 may be easily optimized for different variants of blister pocket geometries or medicament formulations. Separated stacks 2280A, 2280B may be beneficial when it is desired to adjust the relative airflow properties of the first and second entrainment airflow paths 2296, 2298. For example, if the powdered medicament 168A in open blister pocket 164A requires a higher level of turbulence for deagglomeration than the powdered medicament 168B in open blister pocket 164B, the geometry of the path for first entrainment airflow path 2296 may be adjusted to achieve this, in order to maximize the overall efficacy of medicament delivery. Separating the first and second entrainment airflow paths 2296, 2298 through the stacks 2280A, 2280B allows for easier control over the relative airflow properties for each medicament.

As represented in the flowchart of FIG. 24, the inhalation airstream drawn from outside the inhaler device 100 is divided between the two inlets of the manifold 2214, namely the first atrium inlet 2274A and the second atrium inlet 2274B. The inhalation airstream drawn from outside the inhaler device thus simultaneously enters each of the first atrium 2272A and the second atrium 2272B. A first portion of the inhalation airstream entering the first atrium 2272A flows into the open blister pocket 164A and a second portion of the inhalation airstream entering the first atrium 2272A flows into the first stack 2280A. The first portion of the inhalation airstream within the open blister pocket 164A picks up or entrains powdered medicament 168A disposed within the open blister pocket 164A, and then continues into the first stack 2280A. Within the first stack 2280A, the second portion of the inhalation airstream from the first atrium 2272A breaks up or de-agglomerates the powdered medicament 168A entrained within the first portion of the inhalation airstream. Similarly, at the same time, a first portion of the inhalation airstream entering the second atrium 2272B flows into the open blister pocket 164B and a second portion of the inhalation airstream entering the second atrium 2272B flows into the second stack 2280B. The first portion of the inhalation airstream within the open blister pocket 164B picks up or entrains powdered medicament 168B disposed within the open blister pocket 164B, and then continues into the second stack 2280B. Within the second stack 2280B, the second portion of the inhalation airstream from the second atrium 2272B breaks up or de-agglomerates the powdered medicament 168B entrained within the first portion of the inhalation airstream. The airstreams and medicaments within the first and second stacks 2280A, 2280B do not mix together before exiting the manifold 2214 towards a patient's mouth. Airflow through the first atrium 2272A and the open blister pocket 164A is concurrent to airflow through the second atrium 2272B and the open blister pocket 164B.

In the embodiment of FIGS. 22A-25, the first atrium 2272A and the second atrium 2272B are the same or substantially similar in volume or size such that an airflow resistance through each atrium is substantially similar. However, in another embodiment, the first atrium 2272A and the second atrium 2272B are different in volume or size such that a first airflow resistance through the first atrium 2272A is lower than a second airflow resistance through the second atrium 2272B. Different atrium sizes are beneficial when it is desired to adjust the relative airflow properties of the first and second entrainment airflow paths 2296, 2298. Keeping the airstreams entering into the manifold 2214 separate and independent from each other allows the relative airflow properties for each pocket 164A, 164B to be easily adjusted, because the size or volume of the atriums may be optimized for different variants of medicament formulations or blister pocket geometry.

Although the manifold 2214 is shown with two diversion airflow paths 2292, 2294 therethrough, it will be apparent to one of ordinary skill in the art that the manifold 2214 may include additional diversion airflow paths between the respective atrium and stack pairings. For example, rather than providing a single diversion airflow path between each of the first and second atriums 2272A, 2272B and the first and second stacks 2080A, 2080B, respectively, each atrium 2272A, 2272B may include one or more additional outlets to create one or more additional diversion airflow paths directly to its respective stack without departing from the scope of this invention.

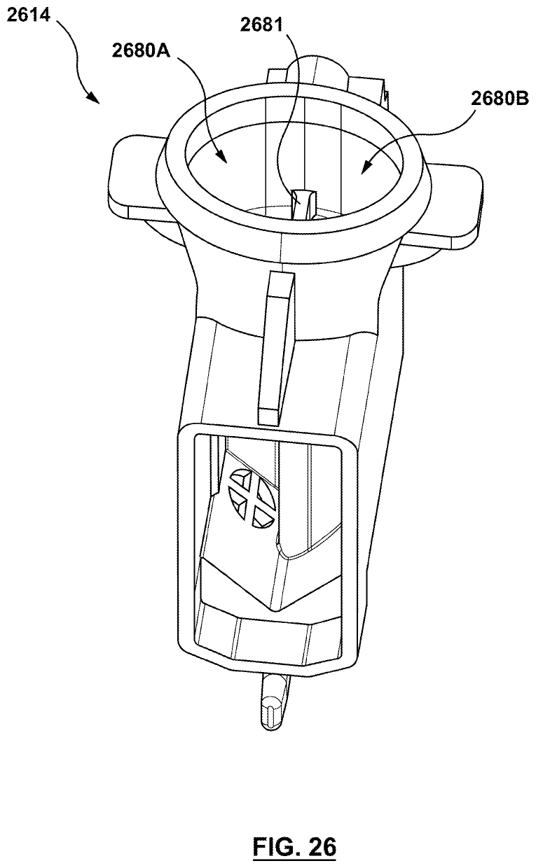

In addition, FIG. 26 depicts a manifold 2614 according to another embodiment hereof. The manifold 2614 is similar to the manifold 2214 except that the manifold 2614 is configured to mix the airstreams within first and second stacks 2680A, 2680B and the respective medicaments therein together before exiting the manifold 2614 towards a patient's mouth, such that a single combined airstream including both medicament 168A from the first blister strip 160A and medicament 168B from the second blister strip 160B exit from the manifold 2614. A divider wall 2681 between the stacks 2680A, 2680B is shortened compared to the divider wall 2281 of the manifold 2214, such that the height of the divider wall 2681 is less than a height of the first and second stacks 2680A, 2680B. When the height of the divider wall 2681 is less than the height of the first and second stacks 2680A, 2680B, mixing of the first entrainment airflow path and the second entrainment airflow path is permitted within the manifold 2614 downstream of the divider wall 2681. Further, the geometry of the divider wall 2681 may be configured or tuned to improve mixing of the first entrainment airflow path and the second entrainment airflow path. For example, the geometry of the divider wall 2681 may direct the first entrainment airflow path and the second entrainment airflow path such that the two airstreams meet at an angle and position which optimizes deagglomeration of the powdered medicaments 168A, 168B.

While various embodiments according to the present invention have been described above, it should be understood that they have been presented by way of illustration and example only, and not limitation. It will be apparent to persons skilled in the relevant art that various changes in form and detail can be made therein without departing from the spirit and scope of the invention. Thus, the breadth and scope of the present invention should not be limited by any of the above-described exemplary embodiments, but should be defined only in accordance with the appended claims and their equivalents. It will also be understood that each feature of each embodiment discussed herein, and of each reference cited herein, can be used in combination with the features of any other embodiment. All patents and publications discussed herein are incorporated by reference herein in their entirety.

1. A manifold for use in an inhaler device for the simultaneous delivery of powdered medicament from an open blister pocket of each of a first blister strip and a second blister strip, the manifold comprising:
a body defining
a first atrium that includes an atrium inlet, a first atrium outlet and a second atrium outlet,
a second atrium that includes an atrium inlet, a first atrium outlet and a second atrium outlet, and
at least one stack that includes at least one stack outlet, a first stack inlet, a second stack inlet, a third stack inlet, and a fourth stack inlet, wherein
the first atrium outlet of the first atrium is in fluid communication with the first stack inlet to define a first diversion airflow path of the manifold,
the first atrium outlet of the second atrium is in fluid communication with the third stack inlet to define a second diversion airflow path of the manifold,
the second atrium outlet of the first atrium is in fluid communication with the second stack inlet to define a first entrainment airflow path associated with an open blister pocket of the first blister strip, and
the second atrium outlet of the second atrium is in fluid communication with the fourth stack inlet to define a second entrainment airflow path associated with an open blister pocket of the second blister strip,
wherein, for each of the first atrium and the second atrium, the first atrium outlet and the second atrium outlet are positioned at opposite sides of the first atrium and second atrium.

2. The manifold of claim 1, wherein the at least one stack is a single stack and the at least one stack outlet is a single stack outlet, and wherein the first diversion airflow path, the second diversion airflow path, the first entrainment airflow path, and the second entrainment airflow path mix together within the single stack.

3. The manifold of claim 2, wherein each of the first and second diversion airflow paths is configured to disrupt each of the first and second entrainment airflow paths and break up powdered medicament carried thereby.

4. The manifold of claim 1, wherein the at least one stack includes a first stack and a second stack, and wherein the first diversion airflow path and the first entrainment airflow path mix together in the first stack, and wherein the second diversion airflow path and the second entrainment airflow path mix together in the second stack.

5. The manifold of claim 4, wherein the first stack and the second stack are separated by a divider wall such that mixing of the first entrainment airflow path and the second entrainment airflow path is prevented within the manifold.

6. The manifold of claim 4, wherein the first stack and the second stack are separated by a divider wall such that mixing of the first entrainment airflow path and the second entrainment airflow path is prevented along a height of the divider wall.

7. The manifold of claim 6, wherein the height of the divider wall is less than a height of the first and second stacks such that mixing of the first entrainment airflow path and the second entrainment airflow path is permitted within the manifold downstream of the divider wall.

8. The manifold of claim 4, wherein the first diversion airflow path is configured to disrupt the first entrainment airflow path and break up powdered medicament carried thereby, and wherein the second diversion airflow path is configured to disrupt the second entrainment airflow path and break up powdered medicament carried thereby.

9. The manifold of claim 1, wherein the first atrium and the second atrium are separated by a divider wall such that the first atrium is not in fluid communication with the second atrium.

10. The manifold of claim 9, wherein the first atrium and the second atrium are substantially similar in volume.

11. The manifold of claim 9, wherein the first atrium and the second atrium are different in volume.

12. The manifold of claim 1, wherein the first atrium and the second atrium are disposed on opposing sides of the body of the manifold.

13. The manifold of claim 1, wherein the first atrium and the second atrium are disposed adjacent to each other on a single side of the body of the manifold.

14. The manifold of claim 1, wherein each of the second atrium outlet of the first atrium and the second atrium outlet of the second atrium includes a grill spanning it.

15. The manifold according to claim 1, wherein the first diversion airflow path, the second diversion airflow path, the first entrainment airflow path, and the second entrainment airflow path are respective airflow portions of an inhalation airflow which is drawn into the manifold through the atrium inlet of each of the first and second atriums when an inhalation force is applied at the at least one stack outlet.

16-27. (canceled)
